(19) United States
(12) Patent Application Publication
(10) Pub. No.: US 2023/0398128 A1
(43) Pub. Date: Dec. 14, 2023

(54) PREFERRED ORAL TESTOSTERONE UNDECANOATE THERAPY TO ACHIEVE TESTOSTERONE REPLACEMENT TREATMENT

(71) Applicant: Marius Pharmaceuticals LLC, Raleigh, NC (US)

(72) Inventors: Om DHINGRA, Morrisville, NC (US); James S. BERNSTEIN, Raleigh, NC (US)

(21) Appl. No.: 18/201,498

(22) Filed: May 24, 2023

Related U.S. Application Data
(63) Continuation of application No. PCT/US2022/029819, filed on May 18, 2022.
(60) Provisional application No. 63/190,609, filed on May 19, 2021.

(51) Int. Cl. A61K 31/568; A61K 47/28; A61K 47/44; A61K 47/34; A61P 9/02; A61K 9/00

(52) U.S. Cl. CPC A61K 31/568; A61K 47/28; A61K 47/44; A61K 47/34; A61P 9/02; A61K 9/0053

(57) ABSTRACT

The present invention features new testosterone undecanoate (TU) dosing regimens, e.g., for testosterone replacement therapy. The TU may be formulated with phytosterols or phytosterol esters.

### BACKGROUND OF THE INVENTION

For testosterone replacement therapy (TRT) in hypogonadal men, the FDA has imposed regulatory guidelines on testosterone (T) formulations to balance the benefits and safety risks associated with abnormally high T concentrations (see, e.g., page 13 of Testosterone Replacement Therapy Advisory Committee Briefing Document, Sep. 17, 2014). These regulatory guidelines include an average T blood serum concentrations (Cavg) in the normal range of 300 to 1000 ng/dL in 75% of subjects, maximum T blood serum concentrations (Cmax) less than 1500 ng/dL in 85% of subjects, not more than 5% between 1800 and 2500 ng/dL, and none above 2500 ng/dL. These guidelines are the standards used to achieve FDA approval to which all pharmaceutical companies attempting to bring testosterone replacement therapies to the market focus their research. To better manage the benefits and safety risks, it is important that formulations and dosing strategies be designed to achieve favorable pharmacokinetic (PK) performance of testosterone or a testosterone prodrug, such as testosterone undecanoate (TU). Accordingly, new formulations, dosage regimens, and titration schemes are needed to meet these standards and improve therapeutic efficacy.

### SUMMARY OF THE INVENTION

The present invention features new testosterone undecanoate dosing strategies that include performing plasma or serum measurements of testosterone and titrating the daily dosage up or down, if necessary, in order to achieve favorable PK parameters.

In one aspect, the invention features a method of treating testosterone deficiency in a subject in need thereof. The subject to be treated is a male, e.g., a hypogonadal male. The method includes performing a treatment regimen that includes administering to the subject a pharmaceutical composition including testosterone undecanoate (TU), a non-sterol solubilizing agent effective for solubilization of the TU, and a phytosterol or phytosterol ester. About 400 mg TU may be administered, e.g., at the onset of the treatment regimen. The method may include establishing a first steady state serum concentration of testosterone. The method may include providing a first Serum Value of testosterone in the subject following administration of the TU. Additionally, the method may further include performing a first titration of the testosterone undecanoate, e.g., if necessary. If the first Serum Value of testosterone is less than about 400/F or 400/F+b ng/dL (e.g., a serum concentration of less than about 449 ng/dL or less than about 460 ng/dL or a plasma concentration of less than about 400 ng/dL), then the daily dosage may be increased, e.g., to about 600 mg TU. F corresponds to a predetermined empirical factor that relates the plasma and serum concentrations and is described in more detail below. This may establish a second steady state Serum Value of testosterone that is higher than the first steady state Serum Value of testosterone. If the first Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the daily dosage may be maintained. This may maintain the first steady state Serum Value of testosterone. If the first Serum Value of testosterone is greater than about 900/F ng/dL or 900/F+b ng/dL (e.g., a serum concentration of greater than about 1011 ng/dL or greater than about 971 ng/dL or a plasma concentration of greater than about 900 ng/dL), then the daily dosage may be decreased, e.g., to about 200 mg TU. This may establish a second steady state Serum Value of testosterone that is lower than the first steady state Serum Value of testosterone.

In another aspect, the invention features a method of treating testosterone deficiency in a subject in need thereof. The subject to be treated may be a male, e.g., a hypogonadal male. The method includes performing a treatment regimen that includes administering to the subject a pharmaceutical composition including testosterone undecanoate (TU), a non-sterol solubilizing agent effective for solubilization of the TU, and a phytosterol or phytosterol ester. About 400 mg TU may be administered, e.g., at the onset of the treatment regimen. The method may include establishing a first steady state serum concentration of testosterone. The method may include providing a first Serum Value of testosterone in the subject following administration of the TU. Additionally, the method may further include performing a first titration of the testosterone undecanoate, e.g., if necessary. If the first Serum Value of testosterone is less than about 460 ng/dL, then the daily dosage may be increased, e.g., to about 600 mg TU. This may establish a second steady state Serum Value of testosterone that is higher than the first steady state Serum Value of testosterone. If the first Serum Value of testosterone is from about 460 ng/dL to about 971 ng/dL, then the daily dosage may be maintained. This may maintain the first steady state Serum Value of testosterone. If the first Serum Value of testosterone is greater than about 971 ng/dL, then the daily dosage may be decreased, e.g., to about 200 mg TU. This may establish a second steady state Serum Value of testosterone that is lower than the first steady state Serum Value of testosterone. The subject may be, for example, on anti-hypertensive therapy and exhibit an average change in systolic blood pressure of no more than 3.4 mmHg, an average change in diastolic blood pressure of no more than 1.8 mmHg, and/or an average change in heart rate of no more than 1.3 beats per minute. The subject may have diabetes mellitus and exhibit an average change in systolic blood pressure of no more than 3.0 mmHg, an average change in diastolic blood pressure of no more than 1.7 mmHg, and/or an average change in heart rate of no more than 1.9 beats per minute.

The Serum Value of testosterone may be measured from about 3 hours to about 6 hours (e.g., 3 hours, 4 hours, 5 hours, or 6 hours, e.g., from about 3 hours to about 5 hours) after administration. The Serum Value of testosterone may be measured from about 3 hours to about 5 hours after administration. The pharmaceutical composition may be administered with a meal. The pharmaceutical composition may be administered in two or more doses (e.g., 2, 3, 4, 5, 6, 7, 8, 9, 10, or more) doses. The pharmaceutical composition may be administered in two doses per day (e.g., twice daily administration). The pharmaceutical composition may be administered in three doses per day. A first dose may be administered in the morning, and a second dose may be administered in the evening. The first dose may include about 200 mg TU, and the second dose may include about 200 mg TU.

In some embodiments, following the first titration: the daily dosage may be increased to about 600 mg TU, and the first dose includes about 300 mg TU, and the second dose includes about 300 mg TU; the daily dosage may be maintained at about 400 mg TU, and the first dose includes about 200 mg TU, and the second dose includes about 200 mg TU; or the daily dosage may be decreased to about 200 mg TU, and the first dose includes about 100 mg TU, and the second dose includes about 100 mg TU.

In some embodiments, a second Serum Value of testosterone may be measured.

In some embodiments, a second titration may be performed, e.g., following the second Serum Value of testosterone measurement.

The first Serum Value of testosterone may be less than about 400/F ng/dL or 400/F+b ng/dL (e.g., a serum concentration of less than about 449 ng/dL or less than about 460 ng/dL or a plasma concentration of less than about 400 ng/dL) and the daily dosage may be increased to about 600 mg TU. This may establish a third steady state Serum Value of testosterone that is higher than the second steady state Serum Value of testosterone. The first Serum Value of testosterone may be from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL) and the dosage may be maintained. This may maintain the second steady state Serum Value of testosterone. The first Serum Value of testosterone may be greater than about 900/F ng/dL or 900/F+b ng/dL (e.g., a serum concentration of greater than about 1011 ng/dL or greater than about 971 ng/dL or a plasma concentration of greater than about 900 ng/dL) and the dosage may be decreased to about 200 mg TU. This may establish a third steady state Serum Value of testosterone that is lower than the first steady state Serum Value of testosterone.

Following the first titration, about 600 mg TU may be administered daily to the subject. If the second Serum Value of testosterone is less than about 400/F ng/dL or 400/F+b ng/dL (e.g., a serum concentration of less than about 449 ng/dL or less than about 460 ng/dL or a plasma concentration of less than about 400 ng/dL), then the method may include orally administering about 800 mg TU daily to the subject to establish a third steady state Serum Value of testosterone that is higher than the second steady state Serum Value of testosterone. If the second Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the method may include continuing to orally administer about 600 mg TU daily to the subject to maintain the second steady state Serum Value of testosterone. If the second Serum Value of testosterone is greater than about 900/F ng/dL or 900/F+b ng/dL (e.g., a serum concentration of greater than about 1011 ng/dL or greater than about 971 ng/dL or a plasma concentration of greater than about 900 ng/dL), then the method may include orally administering about 400 mg TU daily to the subject to establish a third steady state Serum Value of testosterone that is lower than the second steady state Serum Value of testosterone.

Following the first titration, about 400 mg TU may be administered daily to the subject. If the second Serum Value of testosterone is less than about 400/F ng/dL or 400/F+b ng/dL (e.g., a serum concentration of less than about 449 ng/dL or less than about 460 ng/dL or a plasma concentration of less than about 400 ng/dL), then the method may include orally administering about 600 mg TU daily to the subject to establish a third steady state Serum Value of testosterone that is higher than the second steady state Serum Value of testosterone. If the second Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the method may include continuing to orally administer about 400 mg TU daily to the subject to maintain the second steady state Serum Value of testosterone. If the second Serum Value of testosterone is greater than about 900/F ng/dL or 900/F+b ng/dL (e.g., a serum concentration of greater than about 1011 ng/dL or greater than about 971 ng/dL or a plasma concentration of greater than about 900 ng/dL), then the method may include orally administering about 200 mg TU daily to the subject to establish a third steady state Serum Value of testosterone that is lower than the second steady state Serum Value of testosterone.

Following the first titration, about 200 mg TU may be administered daily to the subject. If the second Serum Value of testosterone is less than about 400/F ng/dL or 400/F+b ng/dL (e.g., a serum concentration of less than about 449 ng/dL or less than about 460 ng/dL or a plasma concentration of less than about 400 ng/dL), then the method may include orally administering about 400 mg TU daily to the subject to establish a third steady state Serum Value of testosterone that is higher than the second steady state Serum Value of testosterone. If the second Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the method may include continuing to orally administer about 200 mg TU daily to the subject to maintain the second steady state Serum Value of testosterone. If the second Serum Value of testosterone is greater than about 900/F ng/dL or 900/F+b ng/dL(e.g., a serum concentration of greater than about 1011 ng/dL or greater than about 971 ng/dL or a plasma concentration of greater than about 900 ng/dL), then the method may include orally administering about 100 mg TU daily to the subject to establish a third steady state Serum Value of testosterone that is lower than the second steady state Serum Value of testosterone.

In some embodiments, following the second titration: the dosage may be increased to about 800 mg TU and the first dose includes about 400 mg TU, and the second dose includes about 400 mg TU. In some embodiments, the dosage may be decreased to about 100 mg TU and the subject receives a single dose of about 100 mg TU. The single dose of about 100 mg TU may be administered in the morning.

The first Serum Value of testosterone may be measured once steady state has been achieved. For example, the first Serum Value of testosterone may be measured prior to day 21, e.g., on from about day 1 to about day 21 (e.g., day 1, 2, 3, 4, 5, 6, 7, 8, 9, 10, 11, 12, 13, 14, 15, 16, 17, 18, 19, 20, or 21, e.g., 14) of a treatment regimen. The first Serum Value of testosterone may be measured on from about day 30 to about day 60 (e.g., day 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60) of a treatment regimen.

The first titration may be performed any time after the first Serum Value of testosterone is measured, e.g., on from about day 1 to about day 35, e.g., on from about day 7 to about day 35, e.g., from about day 21 to about day 35 (e.g., day 1, 2, 3, 4, 5, 6, 7, 8, 9, 10, 11, 12, 13, 14, 15, 16, 17, 18, 19, 10 20, 21, 22, 23, 24, 25, 26, 27, 28, 29, 30, 31, 32, 33, 34, or 35, e.g., 28) of the treatment regimen. The first titration may be performed on from about day 30 to about day 60 (e.g., day 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60) of a treatment regimen.

For example, the first Serum Value of testosterone may be measured on about day 14 of the treatment regimen and/or the first titration may be performed on about day 28 of the treatment regimen.

A second Serum Value of testosterone may be measured. For example, the second Serum Value of testosterone may be measure on from about day 35 to about day 49 (e.g., day 35, 36, 37, 38, 39, 40, 41, 42, 43, 44, 45, 46, 47, 48, or 49, e.g., 42) of the treatment regimen.

A second titration may be performed, e.g., following the second Serum Value of testosterone measurement. The second titration may be performed on from about day 49 to about day 63 (e.g., day 49, 50, 51, 52, 53, 54, 55, 56, 57, 58, 59, 60, 61, 62, or 63, e.g., 56) of the treatment regimen. For example, the second Serum Value of testosterone may be measured on about day 42, and the second titration may be formed on about day 56.

In some embodiments, the first titration may be performed on about day 28 of the treatment regimen, and/or the second titration may be performed on about day 56 of the treatment regimen.

In some embodiments, the first titration may be performed, e.g., on from about day 21 to about day 35 (e.g., day 21, 22, 23, 24, 25, 26, 27, 28, 29, 30, 31, 32, 33, 34, or 35, e.g., 28) of the treatment regimen. Following the first titration, the second steady state Serum Value of testosterone may be established. Then, a second Serum Value of testosterone may be measured. A second titration may then be performed.

The subject has not previously been administered TU or other testosterone replacement therapies (e.g., a prodrug of TU) for a period of at least seven days (e.g., 2 weeks, 3 weeks, 4 weeks, 2 months, 3 months, 4 months, 5 months, 6 months, 1 year, or more). For example, the period may be sufficient to wash out all exogenous testosterone from the body.

In some embodiments, the method is performed on a population of human subjects. The population of subjects may include, e.g., at least 10 subjects, at least 50 subjects, at least 100 subjects, at least 200 subjects, at least 500 subjects, or more.

In some embodiments, the method achieves a Cavg in the serum normal range of about 300 ng/dL to about 1000 ng/dL in at least 75% of the population; achieves a Cmax of less than about 1500 ng/dL in at least 85% of the population; achieves a Cmax of from about 1800 ng/dL to about 2500 ng/dL in no more than 5% of the population; and/or achieves a Cmax of greater than about 2500 ng/dL in no more than 0% of the population.

In some embodiments, the method reduces an average number of incorrect titrations or the risk of incorrect titrations per subject in the population in order to achieve a steady state testosterone Serum Value of from about 300 ng/dL to about 1000 ng/dL relative to a population receiving a treatment regimen in which an initial dosage is not about 400 mg TU and/or the Serum Value is not measured from about 3 hours to about 6 hours after administration.

In some embodiments, the method achieves a Cavg in the serum normal range of about 300 ng/dL to about 1000 ng/dL in a greater number of subjects in the population as compared to a treatment regimen in which an initial dosage is not about 400 mg TU and/or the Serum Value is not measured from about 3 hours to about 6 hours after administration; achieves a Cmax of less than about 1500 ng/dL in a greater number of subjects in the population as compared to the treatment regimen in which the initial dosage is not about 400 mg TU and/or the Serum Value is not measured from about 3 hours to about 6 hours after administration; achieves a Cmax of from about 1800 ng/dL to about 2500 ng/dL in a fewer number of subjects in the population as compared to the treatment regimen in which the initial dosage is not about 400 mg TU and/or the Serum Value is not measured from about 3 hours to about 6 hours after administration; and/or achieves a Cmax of greater than about 2500 ng/dL in a fewer number of subjects in the population as compared to the treatment regimen in which the initial dosage is not about 400 mg TU and/or the Serum Value is not measured from about 3 hours to about 6 hours after administration.

In some embodiments, the method decreases the risk of elevated blood pressure, e.g., in the population of human subjects. For example, in some embodiments, daytime systolic blood pressure, night time systolic blood pressure, and/or 24-hour average systolic blood pressure does not increase by more than about 5 mmHg (e.g., no more than about 4, 3, or 2 mmHg) relative to baseline. In some embodiments, daytime systolic blood pressure, night time systolic blood pressure, and/or 24-hour average systolic blood pressure does not increase by more than about 3 mmHg relative to baseline. In some embodiments, daytime systolic blood pressure, night time systolic blood pressure, and/or 24-hour average systolic blood pressure does not increase by more than about 2 mmHg relative to baseline when measured by ambulatory blood pressure monitoring (ABPM). In some embodiments, the subject is diabetic or hypertensive and the daytime systolic blood pressure, night time systolic blood pressure, and/or 24-hour average systolic blood pressure does not increase by more than about 4 mmHg relative to baseline when measured by ambulatory blood pressure monitoring (ABPM).

In some embodiments, the population averages has a Cmax/Cavg ratio for 0-24 hours of less than 2.5; a Cmax/Cavg ratio for 0-12 hours of less than 2.2; and/or a Cmax/Cavg ratio for 12-24 hours of less than 2.2.

The pharmaceutical composition may include from about 5% to about 40% (e.g., about 5%, 10%, 15%, 20%, 25%, 30%, 35%, 40%, e.g., about 18.2%) by weight TU. The pharmaceutical composition may include about from about 2% to about 45% (e.g., about 2%, 5%, 10%, 15%, 20%, 25%, 30%, 35%, 40%, or 45%, e.g., about 25%) by weight of a phytosterol or phytosterol ester. The phytosterol may include phytosterols, phytosterol esters, or combinations thereof. The pharmaceutical composition may include phytosterol esters. The formulation may include from about 10% to about 90% (e.g., about 10%, 15%, 20%, 25%, 30%, 35%, 40%, 45%, 50%, 55%, 60%, 65%, 70%, 75%, 80%, 85%, or 90%) by weight of a non-sterol solubilizing agent. The non-sterol solubilizing agent may be selected from lipids, surfactants (e.g., hydrophobic and/or hydrophilic surfactants), and mixtures thereof. The pharmaceutical composition may be self-emulsifying or self-microemulsifying.

In some embodiments, the non-sterol solubilizing agent includes propylene glycol monolaurate.

In some embodiments, the non-sterol solubilizing agent includes polyoxyl 40 hydrogenated castor oil.

In some embodiments, the pharmaceutical composition includes from about 10% to about 25% (e.g., about 15%, 20%, or 25%, e.g., about 18.2%) by weight of solubilized testosterone undecanoate; from about 5% to about 40% (e.g., about 10%, 15%, 20%, 25%, 30%, 35%, or 40%, e.g., about 15%) by weight of a hydrophilic surfactant; from about 15% to about 65% (e.g., about 20%, 25%, 30%, 35%, 40%, 45%, 50%, 55%, 60%, or 65%, e.g., about 39.9%) by weight of a hydrophobic surfactant; from about 2% to about 45% (e.g., about 2%, 5%, 10%, 15%, 20%, 25%, 30%, 35%, 40%, or 45%, e.g., about 25%) by weight of phytosterol esters; and from about 0 to about 15% (e.g., about 1%, 2%, 3%, 4%, 5%, 10%, or 15%, e.g., about 2%) by weight of a solubilizer.

In some embodiments, oral formulation includes from about 10% to about 40% (e.g., from about 10% to about 30%, e.g., about 25%) by weight of one or more phytosterol esters.

In some embodiments, the solubilizer includes dl-alpha-tocopherol and/or an ester or acetate thereof.

In some embodiments, the pharmaceutical composition includes: about 18.2% by weight of solubilized testosterone undecanoate; about 15.0% by weight of polyoxyl 40 hydrogenated castor oil; about 39.9% by weight of propylene glycol monolaurate; about 25.0% by weight of one or more phytosterol esters; and about 2.0% by weight of dl-alpha-tocopherol and/or an ester or acetate thereof.

In some embodiments of any of the above aspects, the first Serum Value and/or the second Serum Value is measured by measuring testosterone concentration of serum clotted at room temperature for about 30 minutes prior to centrifugation in a tube, measuring testosterone concentration of plasma in a tube supplemented with EDTA and NaF and multiplying the testosterone concentration by the inverse of a predetermined factor F (1/F), or a comparable method thereof. The predetermined factor may be, for example from about 0.70 to about 1.10, e.g., about 0.81 to about 0.94 (e.g., 0.81, 0.82, 0.83, 0.84, 0.85, 0.86, 0.87, 0.88, 0.89, 0.90, 0.91, 0.92, 0.93, or 0.94). For example, the predetermined factor may be 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, or 1.10. In one embodiment, the predetermined factor is 0.82. In one embodiment, the predetermined factor is 0.83. In one embodiment, the predetermined factor is 0.88. In one embodiment, the predetermined factor is 0.89. In another embodiment, the predetermined factor is 0.92.

In some embodiments of any of the above aspects, the subject is at risk of high blood pressure, heart attack, or stroke.

The subject may be suffering from low testosterone levels due to aging. The subject may be suffering from low testosterone levels due to a disease which decreases testosterone production.

The subject may have diabetes (e.g., diabetes mellitus), hypertension, a metabolic disorder, or is obese.

In some embodiments, the subject has been treated or is being treated with an anti-hypertensive medication.

The subject may have osteoporosis, reduced sexual function or libido, muscle strength or muscle stamina, aplastic anemia, AIDS wasting syndrome, obstructive sleep apnea, metabolic disorders, non-alcoholic fatty liver disease (NAFLD), or non-alcoholic steatohepatitis (NASH).

The subject mat be at risk of a testosterone related adverse event (e.g., blood pressure increase).

### Definitions

As used herein, the term “about” refers to a value that is +/−10% of a recited value. For example, a dose of about 400 mg TU refers to a dose that contains from 360 mg to 440 mg of TU. When referring to days, the term about refers to a value of +/−3 days. For example, an event (e.g., a serum T measurement or a dose titration) that occurs on about day 14 may occur from day 11 to day 17.

As used herein, the term “phytosterol” refers to a class of plant sterol molecules, which are naturally occurring compounds found in plant cell membranes. Phytosterols include both plant sterols and stanols. Phytosterols may be derived from any common plant source, such as soy, wood, tall oil, vegetable oil, and the like. Phytosterols include, for example, β-sitosterol, campesterol, stigmasterol, stigmastanol, campestanol, brassicasterol, ergosterol, lupeol, cycloartenol, and the like. Phytosterols also encompasses esterified derivatives thereof, sometimes referred to as phytosterol esters or phytostanol esters. Phytosterol esters are phytosterols esterified with a fatty acid, such as a long chain (e.g., C₆-C₂₄, e.g., C₁₀-C₂₄, e.g., C₁₄-C₂₄) fatty acid, such as octanoic acid, decanoic acid, undecanoic acid, lauric acid, myristic acid, palmitic acid, stearic acid, oleic acid, linoleic acid, and linolenic acid. Phytosterols and their esters may be fully saturated (e.g., hydrogenated). Commercially available phytosterols and phytosterol esters often comprise a mixture of different compounds. For example, CardioAid™ XF phytosterols, sold by ADM, include at least about 95% total plant sterols, about 40-58% β-sitosterol, about 20-30% campesterol, about 14-22% stigmasterol, about 0-6% brassicasterol, and about 0-5% sitostanol. COROWISE® plant sterols, sold by Cargill, include at least about 94% total plant sterols, about 40-58% β-sitosterol, about 20-28% campesterol, and about 16-23% stigmasterol. Tall oil derived phytosterols may include about 36-79% sitosterol, about 6-34% sitostanol, about 4-25% campesterol, and about 0-14% campestanol. Wood derived phytosterols may include about 72% sitosterol, about 8.2% campesterol, about 0.3% stigmasterol, about 0% brassicasterol, about 15.3% sitostanol, and about 1.6% campestanol. Vegetable oil derived phytosterols may include about 45% sitosterol, about 26.8% campesterol, about 19.3% stigmasterol, about 1.6% brassicasterol, about 2.1% sitostanol, and about 0.8% campestanol. Pharmaceutical compositions containing phytosterols or their esters may include one or more of the foregoing components or a mixture thereof. As used herein, the term “phytosterol” or “phytosterols” encompasses both phytosterols and phytosterol esters.

As used herein, “titration” refers to an increase or decrease of the total daily dosage of testosterone undecanoate administered to a subject, typically based on the response of the subject to the exogenous administered testosterone undecanoate. The dosage can be increased or decreased based on the measurement of serum testosterone concentration after a steady state has been achieved.

As used herein, “steady state” refers to the achievement of a stable response in serum total testosterone levels to exogenously administered testosterone undecanoate, typically achieved after at least 7 (e.g., 8, 9, 10, 11, 12, 13, 14, 15, 16, 17, 18, 19, 20, 21, or 28) days following the start of a dosing regimen.

In some embodiments, the titration can also include the adjustment of the way the total dosage is administered such as whether it is administered as two or three doses within a 24-hour period, whether it is administered with a meal, with a meal with a particular fat content, or at a particular hour of the day.

The terms “plasma testosterone concentration” and “serum testosterone concentration” refer to the “total” testosterone concentration which is the sum of the bioavailable testosterone including free and protein-bound testosterone concentrations, in plasma and serum, respectively. As with any bio-analytical measure, for increased consistency the method employed to measure initial serum testosterone levels should be consistent with the method used to monitor and re-measure serum testosterone levels during clinical testing and testosterone therapy for a subject.

As used herein, of the average plasma or serum testosterone concentration can be determined using methods and practices known in the art. For example, the average baseline plasma or serum testosterone concentration of a human male is the arithmetic mean of the total plasma or serum testosterone concentrations, respectively, determined on at least two consecutive time points that are reasonably spaced from each other, for example from about 1 hour to about 168 hours apart. In one example, the serum or plasma testosterone concentration can be determined on at least two consecutive times that are about 12 hours to about 48 hours apart. In another example, the plasma or serum testosterone concentration of the human male can be determined at a time between about 5 o'clock and about 11 o'clock in the morning. Further, the plasma or serum testosterone concentration can be the determined by standard analytical procedures and methods available in the art, such as for example, automated or manual immunoassay methods, liquid chromatography or liquid chromatography-tandem mass spectrometry (LC-MS/MS) and the like.

As used herein, the term “Serum Value” refers to a specified Cavg serum concentration testosterone and a corresponding plasma testosterone concentration. The serum concentration is multiplied by a predetermined factor (F) to convert the serum concentration into the corresponding plasma concentration such that:

F*(serum concentration)=plasma concentration; and

Serum Value=serum concentration=(1/F)*(plasma concentration).

As serum and plasma measurements of testosterone yield different values depending on the assay used for measurement, a predetermined factor F is needed to correlate measurements using different assays. The predetermined factor is calculated empirically and may be from, e.g., from about 0.70 to about 1.10, e.g., from about 0.81 to about 0.94 (e.g., 0.81, 0.82, 0.83, 0.84, 0.85, 0.86, 0.87, 0.88, 0.89, 0.90, 0.91, 0.92, 0.93, or 0.94). The predetermined factor may be 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, or 1.10. In one particular embodiment, the predetermined factor is about 0.89 when the plasma measurement is conducted using plasma sample tubes containing NaF/EDTA. In this embodiment, a Serum Value of about 300 ng/dL refers to a serum concentration of testosterone of about 300 ng/dL and a NaF/EDTA plasma concentration of testosterone of about 267 ng/dL (300 multiplied by 0.89 ng/dL). A Serum Value of about 1000 ng/dL refers to a serum concentration of testosterone of about 1000 ng/dL and a NaF/EDTA plasma concentration of testosterone of about 1000 multiplied by 0.89 ng/dL, which is about 890 ng/dL. The NaF/EDTA plasma concentrations of testosterone used as cutoffs for titration decisions of 400 ng/dL and 900 ng/dL may refer to Serum Values of (400/0.89)=449 ng/dL and (900/0.89)=1011 ng/dL, respectively. One of skill in the art would appreciate that due to the error associated with an empirically derived factor, these values can fluctuate within a reasonable error of, e.g., about +/−0 10%. A Serum Value may be obtained by measuring testosterone concentration of serum clotted at room temperature for about 30 minutes prior to centrifugation in a tube, measuring testosterone concentration of plasma in a tube supplemented with EDTA and NaF and multiplying the testosterone concentration by a predetermined factor F, or a comparable method thereof. Exemplary methods are described, e.g., in Lachance et al. Future Sci OA, FSO55, 2015, hereby incorporated by reference in its entirety.

In another embodiment, a linear regression is used to derive an equation that may be used to relate serum and plasma concentrations such that:

F*(serum concentration−b)=plasma concentration; and

Serum Value=serum concentration=(1/F)*(plasma concentration)+b;

where b is the y-intercept in the linear regression and 1/F is the slope.

As serum and plasma measurements of testosterone yield different values depending on the assay used for measurement, a predetermined linear equation is needed to correlate measurements using different assays. For example, the slope 1/F may be 1.023 and the intercept 50.45 ng/dL. In one particular embodiment, the predetermined slope is about 1.023 and the intercept is 50.45 ng/dL when the plasma measurement is conducted using plasma sample tubes containing NaF/EDTA. In this embodiment, a Serum Value of about 300 ng/dL refers to a serum concentration of testosterone of about 300 ng/dL and a NaF/EDTA plasma concentration of testosterone of about 244 ng/dL (300, minus the intercept 50.45, and the resultant divided by 1.023). A Serum Value of about 1000 ng/dL refers to a serum concentration of testosterone of about 1000 ng/dL and a NaF/EDTA plasma concentration of testosterone of about 928 ng/dL (1000, minus the intercept 50.45, and the resultant divided by 1.023). The NaF/EDTA plasma concentrations of testosterone used as cutoffs for titration decisions of 400 ng/dL and 900 ng/dL may refer to Serum Values of (400*1.023)+50.45=460 ng/dL and (900*1.023)+50.45=971 ng/dL, respectively (see, e.g., FIG. 8). One of skill in the art would appreciate that due to the error associated with an empirically derived factor, these values can fluctuate within a reasonable error of, e.g., about +/−10%. A Serum Value may be obtained by measuring testosterone concentration of serum clotted at room temperature for about 30 minutes prior to centrifugation in a tube, measuring testosterone concentration of plasma in a tube supplemented with EDTA and NaF and using the linear equation to convert to a serum value, or a comparable method thereof.

One of skill in the art would also appreciate that the parameters of the linear equation used may be dependent on the analytical methodology of the assay for testosterone. For example, immunoassay may have different selectivity and parameters suitable for relating serum and plasma concentrations may be similarly obtained.

Additionally, one of skill in the art would also appreciate that more refined equations relating the serum and plasma concentrations may be applied to the empirical relationship. For example, a non-linear equation could be used to describe the relationship of serum and plasma concentrations.

As used herein, the term AUC₀₋ₜ is the area under the curve of a plasma-versus-time graph determined for the analyte from the time 0 to time “t”.

As used herein, the terms “Cavg” or “C_{avg-t}” is determined as the AUC₀₋ₜ divided by a predetermined period of time (t). For example, C_{avg-8h} is the average plasma concentration over a period of 8 hours post-dosing determined by dividing the AUC₀₋₈ value by 8. Similarly, C_{avg-12h} is the average plasma concentration over a period of 12 hours post-dosing determined by dividing the AUC₀₋₁₂ value by 12; C_{avg-24h} is the average plasma concentration over a period of 24 hours post-dosing determined by dividing the AUC₀₋₂₄ₕ value by 24, and so on. Unless otherwise stated, all C_{avg} values are considered to be C_{avg-24h}.

As used herein, “Cₜ” refers to the serum concentration of testosterone at time “t” prior to or after administration of the dosage of the current invention. The time “t” is generally in hours, unless otherwise specified. For example, a Cₜ of “C_{(-2 to 0)} refers to serum testosterone concentration measured in sample collected between the time of about 2 hours before and just immediately prior to dosage administration to the subject tested. Similarly, Cₜ of “C_{(-2 to 4)}” refers to serum testosterone concentration measured in sample collected between the time of about 2 hours and 4 hours after administration of a dosage to the subject tested.

As used herein, a PK parameter (e.g., Cavg or Cmax), may be a parameter that is measured in a population of subjects, e.g., who are treated with a TU formulation, e.g., as part of a clinical trial.

As used herein, a “population of subjects” refers to a group of at least 10 (e.g., at least 15, 20, 25, 30, 35, 40, 45, 50, 60, 70, 80, 90, 100, 150, 200, 250, 300, 350, 400, 450, 500, or more) subjects.

As used herein, a “subject” refers to an animal, such as a human subject. The subject may be a male. The subject may be a hypogonadal male. The subject may have or be at risk of developing high blood pressure, heart attack or stroke. The subject may be suffering from low T levels due to aging. The subject may be suffering from low testosterone levels due to a disease which decreases testosterone production. The subject may have a comorbidity, such as diabetes (e.g., diabetes mellitus), hypertension, and/or obesity. The subject may have been treated or is being treated with an anti-hypertensive medication. The subject may have a metabolic disorder, for example when obesity, hypertension and reduced insulin sensitivity are co-present. The subject may be identified from a titration protocol of the testosterone replacement therapy. The subject may have osteoporosis, reduced sexual function or libido, muscle strength or muscle stamina, aplastic anemia, AIDS wasting syndrome, obstructive sleep apnea, non-alcoholic fatty liver disease (NAFLD), or non-alcoholic steatohepatitis (NASH).

### BRIEF DESCRIPTION OF THE DRAWINGS

FIG. 1 is a graphic representation of Study 1 SOV2012-F1 dose titration in the study. Abbreviations: a.m.=morning; N=number of subjects; p.m.=evening; T3-5=plasma testosterone concentration measured between 3 and 5 hours (+10 min) post-morning dose. ^{a}The investigator and the sponsor will review the data for each individual, and the reason for not responding to treatment will be investigated. Assuming correct compliance with study drug, SOV2012-F1 may be increased to 600 mg a.m., 400 mg p.m. at the investigator's discretion, taking safety into consideration, or subjects may be discontinued from the study as non-responders. Data will be reported in the clinical study report.

FIG. 2 is a graph illustrating the theoretical outcomes (correct or incorrect) of titration decisions of using a single blood draw at different time points including 0, 1.5, 3, 4, 5, 6, 8, 10, or 12 hr after morning dosing to predict the 24-hour T Cavg as compared to the calculated 24-hour T Cavg. For illustrating the approach, this figure uses the commonly accepted serum normal T-range of 300 to 1000 ng/dL.

FIG. 3 is a graph showing percentage of subjects at each time point where use of the blood draw would lead to a correct titration decision (Day 7 and 14 based on 24-hour T Cavg).

FIG. 4 is a graph illustrating percentage of incorrect titration decisions based on a single blood Draw (Day 7 and 14).

FIG. 5 is a graph showing percentage of subjects at each time point where use of the blood draw would lead to a correct titration decision (Day 7 and 14 based on Cmax 0-12).

FIG. 6 is a schematic flow chart showing a titration algorithm as described herein.

FIG. 7 is a graph showing the mean plasma T concentration from 0 to 6 hours post dose on day 14 and day 42.

FIG. 8 is a regression plot of serum versus plasma concentrations using serum-plasma concentration pairs obtained between 3 and 5 hours after morning dose.

FIGS. 9A and 9B are graphs showing hourly ambulatory blood pressure (BP) results at baseline and 120 and 180 days after initiating oral testosterone undecanoate therapy. FIG. 9A depicts the ambulatory systolic BP, and FIG. 9B shows the ambulatory diastolic BP.

FIGS. 10A and 10B are graphs showing cumulative distribution functions of percentage change from baseline to days 120 and 180 in ambulatory blood pressure. FIG. 10A depicts the ambulatory systolic BP, and FIG. 10B shows the ambulatory diastolic BP.

FIG. 11 is a graph showing relationship between serum hemoglobin (g/L) at day 90 of treatment and ambulatory systolic BP at day 120 of treatment. A weak, significant, positive relationship was observed.

FIG. 12 is a graph showing valuation between concentration of the serum testosterone and changes in ambulatory systolic BP at day 120 of treatment. No relationship was observed.

FIG. 13 is a graph showing plasma T from 0-24 hours.

FIG. 14 is a graph showing plasma T with standard deviation (SD) from 0-24 hours.

FIG. 15 is a graph showing plasma and serum T from 0-24 hours.

FIG. 16 is a graph showing plasma TU from 0-24 hours.

FIG. 17 is a graph showing plasma TU with standard deviation (SD) from 0-24 hours.

### DETAILED DESCRIPTION

The present invention features new methods for treating testosterone deficiency. In particular, the invention features testosterone undecanoate (TU) dosing regimens that include administration of TU, performing a plasma or serum measurement of testosterone (T), and titrating the dosage (e.g., increasing or decreasing the dosage) if necessary, in order to achieve favorable pharmacokinetic (PK) parameters. Favorable PK parameters may also be obtained without titrating the dosage. Obtaining favorable PK parameters is necessary to achieve FDA approval for testosterone replacement therapy. Currently, the FDA guidelines for testosterone replacement therapy require testosterone blood serum concentrations (Cavg) in the normal range of 300 to 1000 ng/dL in 75% of subjects, maximum T blood serum concentrations (Cmax) less than 1500 ng/dL in 85% of subjects, not more than 5% between 1800 and 2500 ng/dL, and none above 2500 ng/dL. It should be noted that the FDA guidelines are in fact a guideline, and one of skill in the art would appreciate that they could change or become less rigid. For example, another commonly accepted definition of a normal range is from about 264 ng/dL to about 917 ng/dL. Furthermore, it may be that some subjects do, in fact, exhibit a serum Cmax above 2500 ng/dL. However, the goal is to produce as few subjects as possible above this threshold.

In general, the goal is to design a dosing strategy that reduces the number of titrations and serum T measurements in order to simplify the administration, increase patient compliance, and obtain a serum T concentration in a range consistent with normal subjects (e.g., non-hypogonadal males) and reflective of the FDA guidelines, while providing a safe and efficacious therapy. Furthermore, when a titration is performed, it is also desirable to make a correct titration decision such that you do not have to adjust the dosage one or more times. For example, if one were administered a dosage of TU and a serum T concentration was measured that is below the normal range, then it may be desirable to increase the dosage. However, if the dosage were increased too high and the next serum T concentration measurement is above the normal range, then one may need to decrease the dosage. An additional goal is to reduce unwanted side effects associated with testosterone replacement therapy, such as elevated blood pressure. The methods described herein have been shown to satisfy the foregoing goals by minimizing blood pressure increases that may occur with testosterone replacement therapy and reducing incorrect titration decisions.

The invention also features methods of treating subjects at risk of testosterone related adverse events, such as elevated blood pressure and heart rate, in subjects undergoing testosterone replacement therapy. The subject may have or be at risk of developing high blood pressure, heart attack or stroke. The subject may be suffering from low T levels due to aging. The subject may be suffering from low testosterone levels due to a disease which decreases testosterone production. The subject may have a comorbidity, such as diabetes (e.g., diabetes mellitus), hypertension, and/or obesity. The subject may have been treated or is being treated with an anti-hypertensive medication. The subject may have a metabolic disorder, for example when obesity, hypertension and reduced insulin sensitivity are co-present. The subject may be identified from a titration protocol of the testosterone replacement therapy. The subject may have osteoporosis, reduced sexual function or libido, muscle strength or muscle stamina, aplastic anemia, AIDS wasting syndrome, obstructive sleep apnea, non-alcoholic fatty liver disease (NAFLD), or non-alcoholic steatohepatitis (NASH).

Surprisingly, we discovered that using the starting dosages of TU described herein, measuring the serum or plasma concentration of T from about 3 hours to about 5 hours after a dosing event, and titrating the dosage of TU within a predetermined range following the plasma or serum measurement led to improved PK performance in treated subjects, a higher number of correct titration decisions, a lower number of incorrect titration decisions, and a lower risk of increased blood pressure and/or heart rate. Furthermore, using a NaF/EDTA plasma Cavg concentration range of about 400 ng/dL to about 900 mg/dL (e.g., a serum Cavg concentration range of about 449 ng/dL to about 1011 ng/dL if F is 0.89 or from about 460 ng/dL to about 971 ng/dL if the slope 1/F is 1.023 and b is 50.45) to trigger titration decisions may provide a more favorable outcome as compared to a Cavg concentration range of about 300 ng/dL to about 1000 ng/dL. The preferred starting dosages of TU and particular days on which to perform a serum or plasma measurement and implement a dosage titration are described in more detail below. Also surprisingly, we discovered that the present treatment regimen provides lower risk of increased blood pressure and heart rate. This may negate the need for subsequent blood pressure or heart rate medications required by a subject undergoing testosterone replacement therapy.

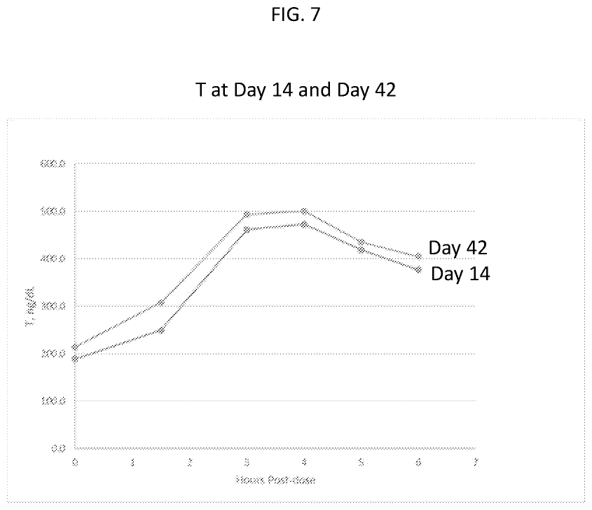

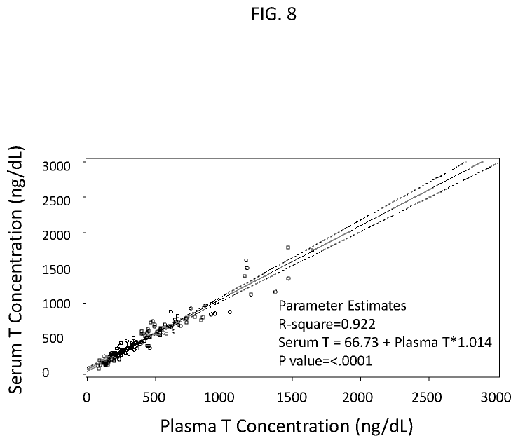

Even further, we also discovered that testosterone values are reliably measured in a window from about 3 hours to about 6 hours (e.g., about 3 hours to about 5 hours) following administration of the TU formulation (e.g., following the morning dose). This window provides a robust measurement window for single measurement evaluation. This feature may be due to the phytosterol esters within the formulation, e.g., due to presence of flat PK curve post dosing, e.g., due to modified release, that permits reliable assessment of subject in the sample window (see FIG. 7). By providing a reliable window in this range, this in turn imparts reliable titration decisions and results, with potential to yield more accurate titration decisions, and reduce unwanted side-effects, such as blood pressure elevation and increased heart rate.

### Dosage and Administration

Described herein are formulations and methods for oral administration of testosterone undecanoate. The oral dosage formulations (e.g., capsule, softgel, tablet, lozenge, syrup, or the like) can be used to treat a subject (e.g., a human, e.g., male human subject). The subject may suffer from testosterone deficiency, such as hypogonadism. Accordingly, the methods described herein provide a serum concentration of testosterone within a target serum testosterone concentration Cave range for a subject (e.g., a male subject) or a population of subjects. The method includes the step of orally administering to the subject a dosage of a pharmaceutical composition containing TU.

The formulation may include TU at about 5% to about 40% (e.g., about 5% to about 35%, about 5% to about 25%, about 5% to about 20%, about 10% to about 35%, about 10% to about 25%, about 10% to about 20%, about 10% to about 15%, about 15% to about 35%, about 15% to about 30%, about 15% to about 25%, about 15% to about 20%) by weight (wt %) of the formulation. For example, the formulation may contain about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 29%, or 40% by weight of the formulation. The pharmaceutical composition may provide a dosage of about 25 mg to about 1000 mg (e.g., about 50 mg to about 600 mg, about 100 mg to about 600 mg, about 200 mg to about 600 mg, about 200 mg to about 400 mg, about 100 mg to about 200 mg) TU per day. For example, the formulation may provide about 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg TU per day.

A pharmaceutical composition may be administered in multiple doses. For example, it is understood that all dosages may be continuously given or divided into multiple doses given per a given time frame. For example, a daily dosage of about 400 mg may be administered in two doses, (e.g., a first dose of about 200 mg and a second dose of about 200 mg, or a first dose of about 100 mg and a second dose of about 300 mg).

The pharmaceutical compositions described herein may be administered one or more times per day. For example, a dose may be administered once per day, twice per day, three times per day, four times per day, five times per day, six times per day, or more. The formulation may be administered with a meal.

### Formulations and Excipients

The formulations used in the methods described herein are provided in a self-emulsifying drug delivery system (SEDDS), self-microemulsifying drug delivery system (SMEDDS), or self-nanoemulsifying drug delivery system (SNEDDS) delivery system, which are known in the art as useful mechanisms for delivery of hydrophobic drugs, such as TU. Hydrophobic drugs are associated with poor water solubility and low oral bioavailability. SEDDS/SMEDDS/SNEDDS formulations are isotropic mixtures of an oil, a surfactant, a cosurfactant (or solubilizer), and a drug. The basic principle of this system is its ability to form fine oil in-water (o/w) microemulsions under gentle agitation following dilution by aqueous phases (e.g., the digestive motility of the stomach and intestine provide the agitation required for self-emulsification in vivo in the lumen of the gut). This spontaneous formation of an emulsion in a fluid environment, such as the gastrointestinal tract presents the drug in a solubilized form, and the small size of the formed droplet provides a large interfacial surface area for drug absorption. Apart from solubilization, the presence of lipid in the formulation further helps improve bioavailability by affecting the drug absorption. Selection of a suitable self-emulsifying formulation depends upon the assessment of the solubility of the drug in various components, the area of the self-emulsifying region as obtained in the phase diagram, the droplet size distribution of the resultant emulsion following self-emulsification, and the release rate of the drug after dispersion in intestinal fluids.

The formulations described herein include TU. TU may be formulated with a non-sterol solubilizing agent, and one or more phytosterols or phytosterol esters. The non-sterol solubilizing agent may include one or more hydrophobic surfactants, one or more hydrophilic surfactants, and/or mixtures thereof.

A lipophilic or hydrophobic surfactant as defined herein is poorly water soluble or water insoluble and has a hydrophilic-lipophilic balance (HLB) value of less than 10, preferably less than 5 and more preferably a HLB of 1 to 3. HLB is an empirical expression for the relationship of the hydrophilic and hydrophobic groups of a surface-active amphiphilic molecule, such as a surfactant. It is used to index surfactants and its value varies from about 1 to about 45 and includes both non-ionic and ionic surfactants. It is well known that the higher the HLB, the more water soluble/dispersible the surfactant.

Exemplary lipophlic surfactants include, but are not limited to, Maisine 35-1, Imwitor 742, Capmul MCM, Capmul PG 12, Lauroglycol 90, Lauroglycol FCC, Caproyl 90, Captex 250, a fatty acid selected from the group consisting of octanoic acid, decanoic acid, undecanoic acid, lauric acid, myristic acid, palmitic acid, stearic acid, oleic acid, linoleic acid, and linolenic acid. Fatty acids may include both a lipophilic and hydrophilic component, and therefore, may be characterized as either a lipophilic or hydrophilic surfactant. As used herein, a lipophilic surfactant may also be referred to as a poorly water-soluble surfactant or a hydrophobic surfactant.

Lipophilic surfactants suitable for use in the formulations described herein include, for example, fatty acids (C₆-C₂₄, e.g., C₁₀-C₂₄, e.g., C₁₄-C₂₄), for example, octanoic acid, decanoic acid, undecanoic acid, lauric acid, myristic acid, palmitic acid, stearic acid, oleic acid, linoleic acid, and linolenic acid.

Lipophilic surfactants suitable for use in the formulations described herein include, for example, mono- and/or di-glycerides of fatty acids, such as Imwitor 988 (glyceryl mono-/di-caprylate), Imwitor 742 (glyceryl mono-/di-caprylate/caprate), Imwitor 308 (glyceryl mono-caprylate), Imwitor 191 (glyceryl mono-stearate), Softigen 701 (glyceryl mono-/di-ricinoleate), Capmul MCM (glyceryl mono-/di-caprylate/caprate), Capmul MCM(L) (liquid form of Capmul MCM), Capmul GMO (glyceryl mono-oleate), Capmul GDL (glyceryl dilaurate), Maisine (glyceryl mono-linoleate), Peceol (glyceryl mono-oleate), Myverol 18-92 (distilled monoglycerides from sunflower oil) and Myverol 18-06 (distilled monoglycerides from hydrogenated soybean oil), Precirol ATO 5 (glyceryl palmitostearate) and Gelucire 39/01 (semi-synthetic glycerides, e.g., 01218 mono-, di- and tri-glycerides).

Lipophilic surfactants suitable for use in the formulations described herein include, for example, acetic, succinic, lactic, citric and/or tartaric esters of mono- and/or di-glycerides of fatty acids, for example, Myvacet 9-45 (distilled acetylated monoglycerides), Miglyol 829 (caprylic/capric diglyceryl succinate), Myverol SMG (mono/di-succinylated monoglycerides), Imwitor 370 (glyceryl stearate citrate), Imwitor 375 (glyceryl monostearate/citrate/lactate) and Crodatem T22 (diacetyl tartaric esters of monoglycerides).

Lipophilic surfactants suitable for use in the formulations described herein include, for example, propylene glycol mono- and/or di-esters of fatty acids, for example, Lauroglycol (propylene glycol monolaurate), Mirpyl (propylene glycol monomyristate), Captex 200 (propylene glycol dicaprylate/dicaprate), Miglyol 840 (propylene glycol dicaprylate/dicaprate) and Neobee M-20 (propylene glycol dicaprylate/dicaprate).

Lipophilic surfactants suitable for use in the formulations described herein include, for example, polyglycerol esters of fatty acids such as Plurol oleique (polyglyceryl oleate), Caprol ET (polyglyceryl mixed fatty acids) and Drewpol 10.10.10 (polyglyceryl oleate).

Lipophilic surfactants suitable for use in the formulations described herein include, for example, castor oil ethoxylates of low ethoxylate content (HLB<10) such as Etocas 5 (5 moles of ethylene oxide reacted with 1 mole of castor oil) and Sandoxylate 5 (5 moles of ethylene oxide reacted with 1 mole of castor oil).

Lipophilic surfactants suitable for use in the formulations described herein include, for example, acid and ester ethoxylates formed by reacting ethylene oxide with fatty acids or glycerol esters of fatty acids (HLB<10) such as Crodet 04 (polyoxyethylene (4) lauric acid), Cithrol 2MS (polyoxyethylene (2) stearic acid), Marlosol 183 (polyoxyethylene (3) stearic acid) and Marlowet G12DO (glyceryl 12 EO dioleate).

Lipophilic surfactants suitable for use in the formulations described herein include, for example, sorbitan esters of fatty acids, for example, Span 20 (sorbitan monolaurate), Crill 1 (sorbitan monolaurate) and Crill 4 (sorbitan mono-oleate).

Lipophilic surfactants suitable for use in the formulations described herein include, for example, transesterification products of natural or hydrogenated vegetable oil triglyceride and a polyalkylene polyol (HLB<10), e.g., Labrafil M1944CS (polyoxyethylated apricot kernel oil), Labrafil M2125CS (polyoxyethylated corn oil), and Gelucire 37/06 (polyoxyethylated hydrogenated coconut).

Lipophilic surfactants suitable for use in the formulations described herein include, for example, alcohol ethyoxylates (HLB<10), e.g., Volpo N3 (polyoxyethylated (3) oleyl ether), Brij 93 (polyoxyethylated (2) oleyl ether), and Marlowet LA4 (polyoxyethylated (4) lauryl ether).

Lipophilic surfactants suitable for use in the formulations described herein include, for example, pluronics, for example, Polyoxyethylene-polyoxypropylene co-polymers and block co-polymers (HLB<10) e.g., Synperonic PE L42 (HLB=8) and Synperonic PE L61 (HLB=3).

In some embodiments, a mixture of lipophilic surfactants, e.g., as described above, may be used in the formulations described herein.

The formulations suitable for use in the methods described herein include any pharmaceutically acceptable hydrophilic surfactant (e.g., having an HLB value greater than 10). Some non-limiting examples include, castor oil or hydrogenated castor oil ethoxylates (HLB>10), e.g., Cremophor EL (polyoxyethylene (35) castor oil), Cremophor RH40 (polyoxyethylene (40) hydrogenated castor oil), Etocas 40 (polyoxyethylene (40) castor oil), Nikkol HCO-60 (polyoxyethylene (60) hydrogenated castor oil), Solutol HS-15 (polyethylene glycol 660 hydroxystearate), Labrasol (caprylocaproyl macrogol-8 glycerides), α-tocopherol-polyethylene glycol-1000-succinate (TPGS) and ascorbyl-6 palmitate. Hydrophilic surfactants suitable for use in the formulations described herein include, for example, polyoxyethylene sorbitan fatty acid derivates, e.g., Tween 20 (polyoxyethylene (20) monolaureate), Tween 80 (polyoxyethylene (20) monooleate), Crillet 4 (polyoxyethylene (20) monooleate) and Montanox 40 (polyoxyethylene (20) monopalmitate).

Hydrophilic surfactants suitable for use in the formulations described herein include, for example, gelucires, preferably Gelucire 50/13 (PEG mono- and diesters of palmitic and stearic acids. (In reference to Gelucires, the first number (e.g., 50) corresponds to the melting point of the material and the second (e.g., 13) to the HLB number.)

Hydrophilic surfactants suitable for use in the formulations described herein include, for example, fatty acid ethoxylates (HLB>10), e.g., Myrj 45 (polyoxyethylene (8) stearate), Tagat L (polyoxyethylene (30) monolaurate), Marlosol 1820 (polyoxyethylene (20) stearate) and Marlosol OL15 (polyoxyethylene (15) oleate). Myrj 45 is preferred.

Hydrophilic surfactants suitable for use in the formulations described herein include, for example, alcohol ethoxylates (HLB>10), e.g., Brij 96 (polyoxyethylene (10) oleyl ether), Volpo 015 (polyoxyethylene (15) oleyl ether), Marlowet OA30 (polyoxyethylene (30) oleyl ether) and Marlowet LMA20 (polyoxyethylene (20) C₁₂-C₁₄ fatty ether).

Hydrophilic surfactants suitable for use in the formulations described herein include, for example, polyoxyethylene-polyoxypropylene co-polymers and block co-polymers (HLB>10), that are commercially available under the trade name Pluronics or Poloxamers, such as Poloxamers 188 and 407 also known as Syperonic PE L44 (HLB=16) and Syperonic F127 (HLB=22), respectively.

Hydrophilic surfactants suitable for use in the formulations described herein include, for example, anionic surfactants, e.g., sodium lauryl sulphate, sodium oleate, and sodium dioctylsulphosuccinate.

Hydrophilic surfactants suitable for use in the formulations described herein include, for example, alkylphenol surfactants (HLB>10), e.g., Triton N-101 (polyoxyethylene (9-10) nonylphenol) and Synperonic NP9 (polyoxyethylene (9) nonylphenol).

In some embodiments, a mixture of hydrophilic surfactants, e.g., as described above, may be used in the formulations described herein.

In some embodiments, a mixture of hydrophilic surfactants and lipophilic surfactants, e.g., as described above, may be used in the formulations described herein.

The formulations described herein may also include one or more additional cosolvents. Cosolvents suitable with the formulations described herein, include, for example, short chain mono-, di-, and polyhydric alcohols, such as ethanol, benzyl alcohol, glycerol, propylene glycol, propylene carbonate, polyethylene glycol with an average molecular weight of about 200 to about 10,000, diethylene glycol monoethyl ether (e.g., Transcutol HP), and combinations thereof. In some embodiments, the formulation further includes water.

The formulations described herein may include an additional oil. Additional oils that may be incorporated in embodiments of the present invention include complete glycerol triesters of medium chain (C₇-C₁₃) or long chain (C₁₄-C₂₂) fatty acids with low molecular weight (up to C₆) mono-, di- or polyhydric alcohols. Some examples of oils for use in this invention thus include: vegetable oils (e.g., soybean oil, safflower seed oil, corn oil, olive oil, castor oil, cottonseed oil, arachis oil, sunflower seed oil, coconut oil, palm oil, rapeseed oil, evening primrose oil, grape seed oil, wheat germ oil, sesame oil, avocado oil, almond, borage, peppermint and apricot kernel oils) and animal oils (e.g., fish liver oil, shark oil, and mink oil).

In some preferred embodiments, the formulations suitable for use in the methods described herein include TU, a non-sterol solubilizing agent, and a phytosterol or phytosterol ester, or a mixture thereof. For example, the formulation may include about 5% to about 40% TU, about 10% to about 90% of a non-sterol solubilizing agent, and about 2% to about 45% by weight of a phytosterol or phytosterol ester. For example, the formulation may include about 5% to about 40% (e.g., about 5% to about 35%, about 5% to about 25%, about 5% to about 20%, about 10% to about 35%, about 10% to about 25%, about 10% to about 20%, about 10% to about 15%, about 15% to about 35%, about 15% to about 30%, about 15% to about 25%, about 15% to about 20%) TU by weight of the formulation. The formulation may include about 10% to about 90% (e.g., about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 90%, about 30% to about 80%, about 30% to about 70%, about 30% to about 60%, about 30% to about 50%, about 30% to about 40%, about 40% to about 90%, about 40% to about 80%, about 40% to about 70%, about 40% to about 60%, about 40% to about 50%, about 50% to about 90%, about 50% to about 80%, about 50% to about 70%, about 50% to about 60%, about 60% to about 90%, about 60% to about 80%, about 60% to about 70%, about 70% to about 90%, about 70% to about 80%, or about 80% to about 90%) by weight of a non-sterol solubilizing agent. In some embodiments, the formulation may include about 10%, about 15%, about 20%, about 25%, about 30%, about 35%, about 40%, about 45%, about 50%, about 55%, about 60%, about 65%, about 70%, about 75%, about 80%, about 85%, or about 90% by weight of a non-sterol solubilizing agent. The formulation may include about 2% to about 45% by weight of a phytosterol or phytosterol ester, or a mixture thereof. For example, the formulation may include about 5% to about 35%, about 5% to about 25%, about 5% to about 20%, about 10% to about 35%, about 10% to about 25%, about 10% to about 20%, about 10% to about 15%, about 15% to about 35%, about 15% to about 30%, about 15% to about 25%, about 15% to about 20% by weight of a phytosterol or phytosterol ester. In some embodiments, the formulation includes about 5%, about 10%, about 15%, about 20%, about 25%, about 30%, about 35%, about 40%, or about 45% by weight of a phytosterol or phytosterol ester or a mixture thereof. The phytosterol can be selected from β-sitosterol, campesterol, stigmasterol, stigmastanol, campestanol, brassicasterol, ergosterol, lupeol, and cycloartenol. Similarly, the phytosterol ester can be a fatty acid ester of a phytosterol selected from β-sitosterol, campesterol, stigmasterol, stigmastanol, campestanol, brassicasterol, ergosterol, lupeol, and cycloartenol.

In some embodiments, the formulation includes from about 10% to about 25% (e.g., about 15% to about 25%, e.g., about 18.2%) by weight of solubilized testosterone undecanoate; from about 5 to about 40% (e.g., about 5% to about 30%, about 10% to about 20%, e.g., about 15.0%) by weight of a hydrophilic surfactant; from about 15% to about 65% (e.g., about 20% to about 60%, about 30% to about 50%, e.g., about 39.9%) by weight of a hydrophobic surfactant; from about 2% to about 45% (e.g., about 5% to about 40%, about 10% to about 30%, e.g., about 25.0%) about by weight of phytosterol esters; and from about 0 to about 15% (e.g., about 0 to about 10%, e.g., about 0 to about 5%, e.g., about 2.0%) by weight of a solubilizer.

In some embodiments, the hydrophilic surfactant is polyoxyl 40 hydrogenated castor oil (e.g., Cremophor RH40). In some embodiments, the hydrophilic surfactant is propylene glycol monolaurate (e.g., Lauroglycol 90). In some embodiments, the solubilizer is dl-alpha tocopherol (e.g., vitamin E) and/or an ester or acetate thereof. In some embodiments, the formulation includes about 18.2% by weight of solubilized testosterone undecanoate; about 15.0% by weight of polyoxyl 40 hydrogenated castor oil; about 39.9% by weight of propylene glycol monolaurate; about 25.0% by weight of one or more phytosterol esters; and about 2.0% by weight of dl-alpha-tocopherol and/or an ester or acetate thereof.

### Titration

The methods described herein include adjusting a dosage of TU in order to optimize one or more PK parameters. The methods include administering to the subject a pharmaceutical composition including testosterone undecanoate (TU), a non-sterol solubilizing agent effective for solubilization of the TU, and a phytosterol or phytosterol ester. The subject has not previously been administered TU or other testosterone replacement therapies (e.g., a prodrug of TU) for a period of at least seven days (e.g., 2 weeks, 3 weeks, 4 weeks, 2 months, 3 months, 4 months, 5 months, 6 months, 1 year, or more). For example, the period may be sufficient to wash out all exogenous testosterone from the body.

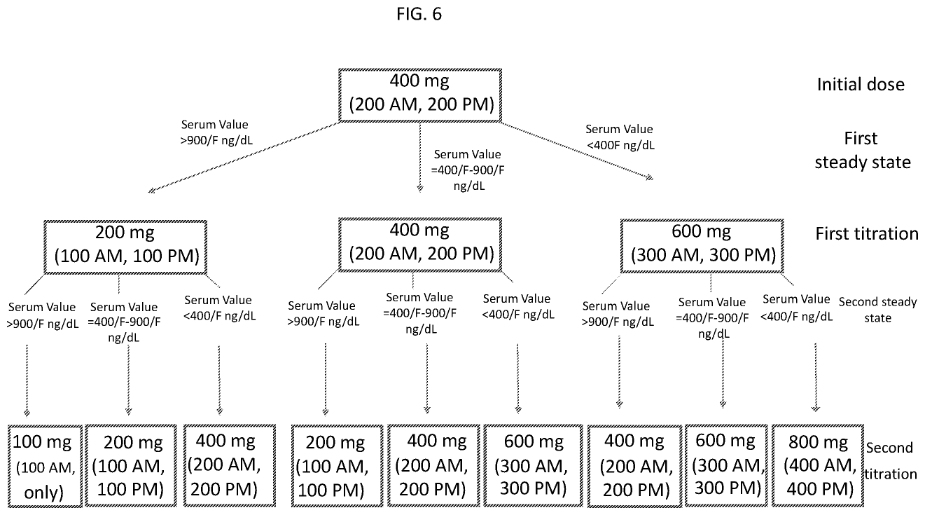

The initial dosage of TU may be from about 100 mg to about 1000 mg TU (e.g., about 200 mg, 300 mg, 400 mg, 500 mg, 600 mg, 700 mg, 800 mg, 900 mg, or 1000). In some embodiments, the initial dosage is about 400 mg. This may be administered daily until a first steady state serum concentration of testosterone is achieved. The method may include providing a first Serum Value of testosterone in the subject following administration of the TU. Additionally, the method may further include performing a first titration of the testosterone undecanoate, e.g., if necessary. If the first Serum Value of testosterone is less than about 400/F ng/dL or 400/F+b ng/dL (e.g., a serum concentration of less than about 449 ng/dL or less than about 460 ng/dL or a plasma concentration of about 400 ng/dL), then the dosage may be increased, e.g., by about 25%, 50%, 100%, 150%, 200%, or more. For example, if the initial dosage is about 400 mg, then the dosage may be increased, e.g., to about 600 mg TU. This may establish a second steady state Serum Value of testosterone that is higher than the first Serum Value of testosterone. If the first Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the dosage may be maintained, e.g., at about 400 mg. This may maintain the first steady state Serum Value of testosterone. If the first Serum Value of testosterone is greater than about 900/F ng/dL or 900/F+b ng/dL (e.g., a serum concentration of greater than about 1011 ng/dL or greater than about 971 ng/dL or a plasma concentration of greater than about 900 ng/dL), then the dosage may be decreased, e.g., by about 25%, 50%, 100%, 150%, 200%, or more. For example, if the initial dosage is about 400 mg, then the dosage may be decreased, e.g., to about 200 mg TU. This may establish a second steady state Serum Value of testosterone that is lower than the first Serum Value of testosterone (see FIG. 6).

When a titration is performed, the dosage of TU may be increased, decreased, or maintained. The dosage may increase by about 50 mg, 100 mg, 150 mg, 200 mg, 250 mg, or 300 mg. The dosage may decrease by about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

A serum or plasma concentration measurement may be performed any time following initiation of TU treatment. For example, a serum or plasma concentration may be measured 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, or more following initiation of TU administration during a treatment regimen.

A titration may be performed any time following an initial administration of TU during a treatment regimen. A titration may be in response to a serum or plasma concentration measurement that occurs following an initial administration of TU during a treatment regimen. For example, a titration may be performed 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, or more following a serum or plasma concentration measurement. In some embodiments, a titration may be performed 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, or more following an initial administration of TU during a treatment regimen.

The Serum Value of testosterone may be measured after administration of TU. For example, the plasma or serum T concentration may be measured from about 3 hours to about 5 hours (e.g., 3 hours, 4 hours, or 5 hours) after administration. The plasma or serum T concentration may be measured after the morning dose. In some embodiments, the plasma or serum T concentration may be measured from about 3 hours to about 6 hours after administration. The pharmaceutical composition may be administered with a meal. Alternatively, the pharmaceutical composition may be administered without a meal. The pharmaceutical composition may be administered in two or more (e.g., 2, 3, 4, 5, 6, 7, 8, 9, 10, or more) doses. The pharmaceutical composition may be administered in two doses per day (e.g., twice daily administration). A first dose may be administered in the morning, and a second dose may be administered in the evening. The doses may be equal. Alternatively, the doses may be different. For example, when administered a daily dosage of about 400 mg TU, the first dose may include about 200 mg TU, and the second dose may include about 200 mg TU.

In some embodiments, following the first titration: the dosage may be increased to about 600 mg TU, and the first dose includes about 300 mg TU, and the second dose includes about 300 mg TU; the dosage may be maintained at about 400 mg TU, and the first dose includes about 200 mg TU, and the second dose includes about 200 mg TU; or the dosage may be decreased to about 200 mg TU, and the first dose includes about 100 mg TU, and the second dose includes about 100 mg TU. In some embodiments, the method further includes providing a second Serum Value of testosterone.

In some embodiments, the method further includes performing a second titration, e.g., following the second Serum Value of testosterone measurement.

Following the first titration, about 600 mg TU may be administered daily to the subject. If the second Serum Value of testosterone is less than about 400/F ng/dL or 400/F+b ng/dL (e.g., a serum concentration of less than about 449 ng/dL or less than about 460 ng/dL or a plasma concentration of less than about 400 ng/dL), then the method may include orally administering about 800 mg TU daily to the subject to establish a third steady state Serum Value of testosterone that is higher than the second steady state Serum Value of testosterone. If the second Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the method may include continuing to orally administer about 600 mg TU daily to the subject to maintain the second steady state Serum Value of testosterone. If the second Serum Value of testosterone is greater than about 900/F ng/dL or 900/F+b ng/dL (e.g., a serum concentration of greater than about 1011 ng/dL or greater than about 971 ng/dL or a plasma concentration of greater than about 900 ng/dL), then the method may include orally administering about 400 mg TU daily to the subject to establish a third steady state Serum Value of testosterone that is lower than the second steady state Serum Value of testosterone.

Following the first titration, about 400 mg TU may be administered daily to the subject. If the second Serum Value of testosterone is less than about 400/F ng/dL or 400/F+b ng/dL (e.g., a serum concentration of less than about 449 ng/dL or less than about 460 ng/dL or a plasma concentration of less than about 400 ng/dL), then the method may include orally administering about 600 mg TU daily to the subject to establish a third steady state Serum Value of testosterone that is higher than the second steady state Serum Value of testosterone. If the second Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the method may include continuing to orally administer about 400 mg TU daily to the subject to maintain the second steady state Serum Value of testosterone. If the second Serum Value of testosterone is greater than about 900/F ng/dL or 900/F+b ng/dL (e.g., a serum concentration of greater than about 1011 ng/dL or greater than about 971 ng/dL or a plasma concentration of greater than about 900 ng/dL), then the method may include orally administering about 200 mg TU daily to the subject to establish a third steady state Serum Value of testosterone that is lower than the second steady state Serum Value of testosterone.

Following the first titration, about 200 mg TU may be administered daily to the subject. If the second Serum Value of testosterone is less than about 400/F ng/dL or 400/F+b ng/dL (e.g., a serum concentration of less than about 449 ng/dL or less than about 460 ng/dL or a plasma concentration of less than about 400 ng/dL), then the method may include orally administering about 400 mg TU daily to the subject to establish a third steady state Serum Value of testosterone that is higher than the second steady state Serum Value of testosterone. If the second Serum Value of testosterone is from about 400/F ng/dL to about 900/F ng/dL or from about 400/F+b ng/dL to about 900/F+b ng/dL (e.g., a serum concentration of from about 449 ng/dL to about 1011 ng/dL or from about 460 ng/dL to about 971 ng/dL or a plasma concentration of from about 400 ng/dL to about 900 ng/dL), then the method may include continuing to orally administer about 200 mg TU daily to the subject to maintain the second steady state Serum Value of testosterone. If the second Serum Value of testosterone is greater than about 900/F ng/dL or 900/F+b ng/dL (e.g., a serum concentration of greater than about 1011 ng/dL or greater than about 971 ng/dL or a plasma concentration of greater than about 900 ng/dL), then the method may include orally administering about 100 mg TU daily to the subject to establish a third steady state Serum Value of testosterone that is lower than the second steady state Serum Value of testosterone.

In some embodiments, following the second titration: the dosage may be increased to about 800 mg TU and the first dose includes about 400 mg TU, and the second dose includes about 400 mg TU; or the dosage may be decreased to about 100 mg TU and the subject receives a single dose of about 100 mg TU. The single dose of about 100 mg TU may be administered in the morning or in the evening.

The first Serum Value of testosterone may be measured once steady stage has been achieved. For example. The first Serum Value of testosterone may be measured from day 1, e.g., on from about day 1 to about day 21, e.g., on from about day 7 to about day 21 (e.g., day 1, 2, 3, 4, 5, 6, 7, 8, 9, 10, 11, 12, 13, 14, 15, 16, 17, 18, 19, 20, or 21, e.g., 14) of a treatment regimen.

The first titration may be performed any time after the first Serum Value of testosterone is measured, e.g., on from about day 1 to about day 35, e.g., on from about day 7 to about day 35, e.g., on from about day 21 to about day 35 (e.g., day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35, e.g., 28) of the treatment regimen. The first titration may be performed on from about day 30 to about day 60 (e.g., day 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60) of a treatment regimen.

For example, the first Serum Value of testosterone may be measured on about day 14 of the treatment regimen and/or the first titration may be performed on about day 28 of the treatment regimen.

A second Serum Value of testosterone may be measured following the first titration, e.g., once a second steady state Serum Value has been achieved. For example, the second Serum Value of testosterone may be measure on from about day 35 to about day 49 (e.g., day 35, 36, 37, 38, 39, 40, 41, 42, 43, 44, 45, 46, 47, 48, or 49, e.g., 42) of the treatment regimen. A second titration may be performed, e.g., following the second Serum Value of testosterone measurement. The second titration may be performed on from about day 49 to about day 63 (e.g., day 49, 50, 51, 52, 53, 54, 55, 56, 57, 58, 59, 60, 61, 62, or 63, e.g., 56) of the treatment regimen. For example, the second Serum Value of testosterone may be measured on about day 42, and the second titration may be formed on about day 56.

In some embodiments, the first titration may be performed on about day 28 of the treatment regimen, and/or the second titration may be performed on about day 56 of the treatment regimen.

In some embodiments, the first titration may be performed, e.g., on from about day 21 to about day 35 (e.g., day 21, 22, 23, 24, 25, 26, 27, 28, 29, 30, 31, 32, 33, 34, or 35, e.g., 28) of the treatment regimen. Following the first titration, the second steady state Serum Value of testosterone may be established. Then, a second Serum Value of testosterone may be measured. A second titration may then be performed.

In some embodiments, the method decreases the risk of elevated blood pressure. For example, in some embodiments, daytime systolic blood pressure, night time systolic blood pressure, and/or 24-hour average systolic blood pressure does not increase by more than about 5 mmHg (e.g., no more than about 4, 3, or 2 mmHg) relative to baseline. In some embodiments, daytime systolic blood pressure, night time systolic blood pressure, and/or 24-hour average systolic blood pressure does not increase by more than about 3 mmHg relative to baseline. In some embodiments, daytime systolic blood pressure, night time systolic blood pressure, and/or 24-hour average systolic blood pressure does not increase by more than about 2 mmHg relative to baseline when measured by ambulatory blood pressure monitoring (ABPM). In some embodiments, the subject is diabetic or hypertensive and the daytime systolic blood pressure, night time systolic blood pressure, and/or 24-hour average systolic blood pressure does not increase by more than about 4 mmHg relative to baseline when measured by ambulatory blood pressure monitoring (ABPM). In some embodiments, the first Serum Value and/or the second Serum Value is measured by measuring testosterone concentration of serum clotted (e.g., at room temperature, e.g., for about 30 to about 50 minutes) prior to centrifugation in a tube, measuring testosterone concentration of plasma in a tube supplemented with EDTA and NaF and multiplying the testosterone concentration by the inverse of a predetermined factor F (1/F), or a comparable method thereof, such as an immunoassay. In some embodiments K2/EDTA tubes or other plasma tubes may be used.

In some embodiments, the method includes performing a treatment regimen that includes administering to the subject a pharmaceutical composition including testosterone undecanoate (TU), a non-sterol solubilizing agent effective for solubilization of the TU, and a phytosterol or phytosterol ester. About 400 mg TU may be administered, e.g., at the onset of the treatment regimen. The method may include establishing a first steady state serum concentration of testosterone. The method may include providing a first Serum Value of testosterone in the subject following administration of the TU. Additionally, the method may further include performing a first titration of the testosterone undecanoate, e.g., if necessary. If the first Serum Value of testosterone is less than about 460 ng/dL, then the daily dosage may be increased, e.g., to about 600 mg TU. This may establish a second steady state Serum Value of testosterone that is higher than the first steady state Serum Value of testosterone. If the first Serum Value of testosterone is from about 460 ng/dL to about 971 ng/dL, then the daily dosage may be maintained. This may maintain the first steady state Serum Value of testosterone. If the first Serum Value of testosterone is greater than about 971 ng/dL, then the daily dosage may be decreased, e.g., to about 200 mg TU. This may establish a second steady state Serum Value of testosterone that is lower than the first steady state Serum Value of testosterone. The subject may be, for example, on anti-hypertensive therapy and exhibit an average change in systolic blood pressure of no more than 3.4 mmHg, an average change in diastolic blood pressure of no more than 1.8 mmHg, and/or an average change in heart rate of no more than 1.3 beats per minute. The subject may have diabetes mellitus and exhibit an average change in systolic blood pressure of no more than 3.0 mmHg, an average change in diastolic blood pressure of no more than 1.7 mmHg, and/or an average change in heart rate of no more than 1.9 beats per minute.

The foregoing titration scheme may be advantageous over other titration schemes. For example, a starting dosage of about 400 mg TU may be advantageous over other starting dosages of TU, such as 800 mg, 700 mg, 600 mg, 500 mg, 300 mg, 200 mg, or 100 mg. A starting dosage of about 400 mg TU may be advantageous over a starting dosage of about 600 mg TU. A starting dosage of about 400 mg TU may be advantageous over a starting dosage of about 200 mg TU. Furthermore, providing a Serum Value of testosterone on from about day 1 to about day 21 (e.g., day 1, 2, 3, 4, 5, 6, 7, 8, 9, 10, 11, 12, 13, 14, 15, 16, 17, 18, 19, 20, or 21, e.g., 14) and/or on from about day 35 to about day 49 (e.g., day 35, 36, 37, 38, 39, 40, 41, 42, 43, 44, 45, 46, 47, 48, or 49, e.g., 42) of the treatment regimen may be advantageous over providing a Serum Value of testosterone during days outside of these ranges or particular days. Performing a titration on from about day 1 to about day 35 (e.g., day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35, e.g., 28) and/or on from about day 49 to about day 63 (e.g., day 49, 50, 51, 52, 53, 54, 55, 56, 57, 58, 59, 60, 61, 62, or 63, e.g., 56) of the treatment regimen may be advantageous over performing a titration during days outside of these ranges or particular days. Furthermore, using a NaF/EDTA plasma Cavg concentration range of about 400 ng/dL to about 900 mg/dL or a serum Cavg concentration range of about 449 ng/dL to about 1011 ng/dL (e.g., if F is 0.89) or from about 460 ng/dL to about 971 ng/dL (e.g., if the slope 1/F is 1.023 and b is 50.45) to trigger titration decisions may provide a more favorable outcome as compared to a Cavg concentration range of about 300 ng/dL to about 1000 ng/dL. Advantageous properties of using the starting dosages, Cavg boundaries, and days on which to measure a serum or plasma concentration of testosterone and/or perform a titration of the TU dosage described herein include increasing correct titration decisions, decreasing incorrect titration decisions, decreasing the risk of titrations, obtaining a population of subjects in which a greater number of subjects fall within the desired FDA guidelines (e.g., serum Cavg in the normal range of 300 to 1000 ng/dL in 75% of subjects, a Cmax less than 1500 ng/dL in 85% of subjects, not more than 5% between 1800 and 2500 ng/dL, and none above 2500 ng/dL), increasing patient compliance, reducing blood pressure, lowering the risk of increasing blood pressure, and increasing responsivity to the testosterone replacement therapy in order to treat the testosterone deficiency in the subject in need thereof.

In some embodiments, the population averages have a Cmax/Cavg ratio for 0-24 hours of less than 2.5; a Cmax/Cavg ratio for 0-12 hours of less than 2.2; and/or a Cmax/Cavg ratio for 12-24 hours of less than 2.2.

### EXAMPLES

### Example 1. High Dose Titration Scheme

The dose of study drug was titrated during the efficacy period using an algorithm that was developed using 24-hr PK data obtained from the 84-day Phase 2b study of SOV2012-F1 in 36 subjects. The final dose established in the 90-day efficacy period for SOV2012-F1 was used at the start of the 9-month safety evaluation period, and the dose was up- or down-titrated on Days 180 and 270 based on the plasma T concentration from a single blood draw within 3 to 5 hours after dosing (Day 166 and Day 256). Subjects on AndroGel were up- or down-titrated on Day 180 and Day 270 based on single-draw serum T Cpredose levels at Day 166 and Day 256, per product information.

Dose reduction occurred for safety based on hemoglobin levels >18 g/dL nominally measured at Days 90, 180, and 270 during the study.

SOV2012-F1 Group

Dose titration for each subject (starting dose was 400 mg TU a.m. and 200 mg TU p.m.) was based on the plasma T measured between 3 to 5 hours (+10 min) after the morning dose at Day 14 and Day 42. Dose titrations occurred at Day 28 and Day 56, if needed, based on the following algorithm:
For subjects who may need dose titration at Day 28 based on the plasma T level obtained between 3 to 5 hours on Day 14:
   T3-5<235 ng/dL: dose increased to 800 mg (400 mg a.m., 400 mg p.m.)
   T3-5≥235 to 1120 ng/dL: no dose change
   T3-5>1120 ng/dL: dose decreased to 400 mg (200 mg a.m., 200 mg p.m.)
For subjects who may need dose titration at Day 56, based on the plasma T level obtained between 3 to 5 hours on Day 42:
   For subjects whose dose was not titrated previously (e.g., remained at 400 mg a.m., 200 mg p.m.) and the resulting plasma T3-5 at Day 42 are:
      T3-5<235 ng/dL: dose increased to 800 mg (400 mg a.m., 400 mg p.m.)
      T3-5≥235 to 1120 ng/dL: no dose change
      T3-5>1120 ng/dL: dose decreased to 400 mg (200 mg a.m., 200 mg p.m.)
   For subjects whose dose was previously decreased to 400 mg (200 mg a.m., 200 mg p.m.), and the resulting plasma T3-5 at Day 42 are:
      T3-5<235 ng/dL: dose increased to 600 mg (400 mg a.m., 200 mg p.m.)
      T3-5≥235 to 1120 ng/dL: no dose change
      T3-5>1120 ng/dL: dose may be further decreased to 200 mg a.m.
   For subjects whose dose was previously increased to 800 mg (400 mg a.m., 400 mg p.m.), and the resulting plasma T3-5 at Day 42 are:
      T3-5<235 ng/dL: The investigator and sponsor will review the data for each individual, and the reason for not responding to the treatment will be further investigated. Assuming correct compliance with the study drug, the dose may be increased to 1000 mg (600 mg a.m., 400 mg p.m.) at the investigator's discretion, taking safety into consideration, or subjects may be discontinued from the study as non-responders.
      T3-5≥235 to 1120 ng/dL: no dose change
      T3-5>1120 ng/dL: dose decreased to 600 mg (400 mg a.m., 200 mg p.m.)

If analysis of the Day 90 24-hour PK data reveals that a subject is on an incorrect dose, discontinuation of the subject may be appropriate.

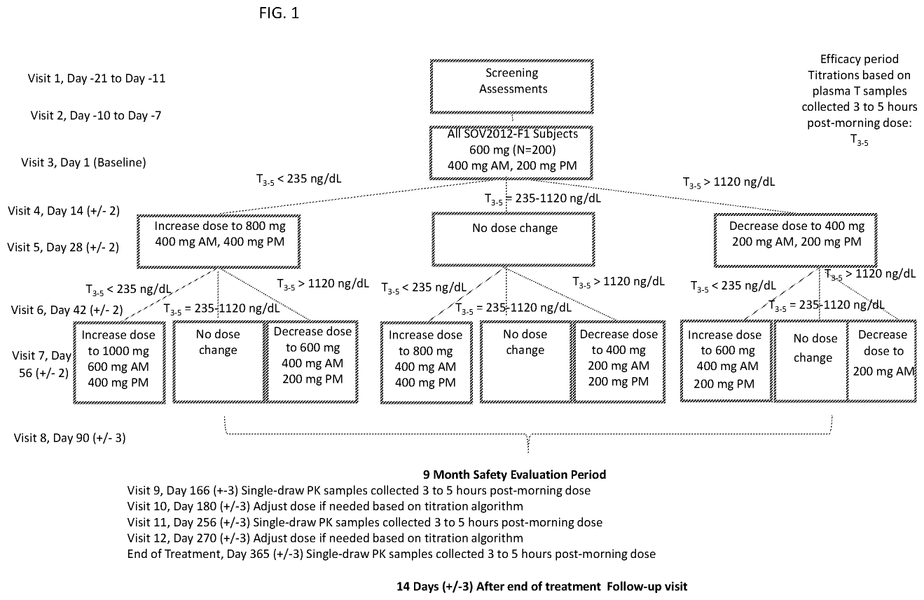

During the 9-month safety evaluation period, the dose was up- or down-titrated on Days 180 and 270 using a single time point T measurement obtained 3 to 5 hours after the morning dose on Days 166, and 256, respectively. A graphic representation of SOV2012-F1 dose titration in the study is provided in FIG. 1.

Methods:

We utilized a similar algorithm from the Axiron product clinical pharmacology review to derive the single blood draw scheme based on data from our Phase Ilb trial.

Briefly, we performed comparisons between the titration recommendation made based on the total plasma T concentration (Cx) from a single blood draw and the titration recommendation made based on 24-hour T Cavg or Cmax.

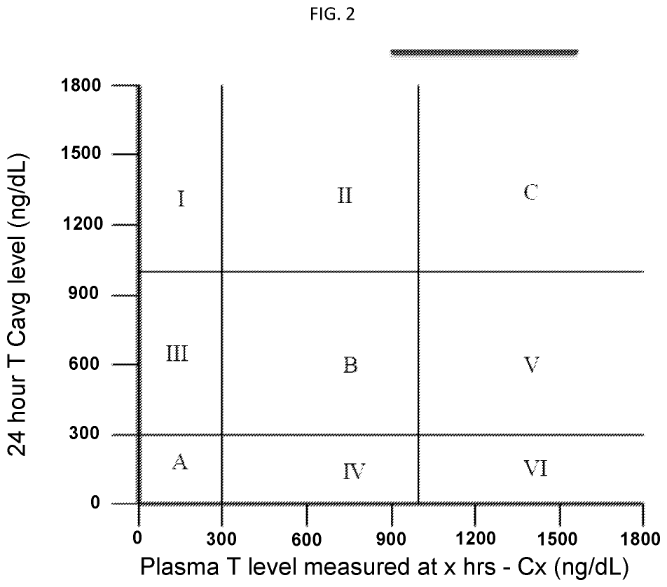

FIG. 2 represents the theoretical outcomes (correct or incorrect titration decisions) of using a single blood draw at different time points including 0, 1.5, 3, 4, 5, 6, 8, 10, or 12 hr after morning dosing to predict the 24-hour T Cavg as compared to the calculated 24-hour T Cavg. For illustrating the approach, this figure uses the commonly accepted serum normal T-range of 300 to 1000 ng/dL.

The regions having discrepancies between Cx-based and 24-hour T Cavg-based titration recommendations are defined as “Incorrect” (e.g., regions I-VI), while regions that both titration recommendations agreed are defined as “Correct” (e.g., regions A, B, and C).

The percentage of subjects within A, B, and C regions represent the correct titration decisions made from single blood draw plasma T levels; while percentage of subjects within regions I-VI represent incorrect decisions as described as following:

I: Plasma T level less than 300 ng/dL, but the Cavg greater than 1000 ng/dL

II: Plasma T level in the normal range, but the Cavg greater than 1000 ng/dL

III: Plasma T level less than 300 ng/dL, but the Cavg in the normal range

IV: Plasma T level in the normal range, but the Cavg less than 300 ng/dL

V: Plasma T level greater than 1000 ng/dL, but the Cavg in the normal range

VI: Plasma T level greater than 1000 ng/dL, but the Cavg less than 300 ng/dL

In situations described in I, II, and III, the single blood draw based titration recommendation will result in a dose higher than necessary while in situations described in IV, V, and VI, the single blood draw based titration recommendations will result in a dose lower than necessary. The same framework applies to Cmax vs. Cx comparison.

### RESULTS

Cavg-Based Decisions

Based on comparisons of 24-hour T Cavg-based titration or Cmax-based titration decisions from Days 7+14 of the Phase 2b study combined (400 mg am/400 mg pm regimen only), we found 0, 1.5, 10 and 12 hours were not suitable for making titration decisions while 3-8 hr post morning dose seemed to be appropriate range for single blood draw.

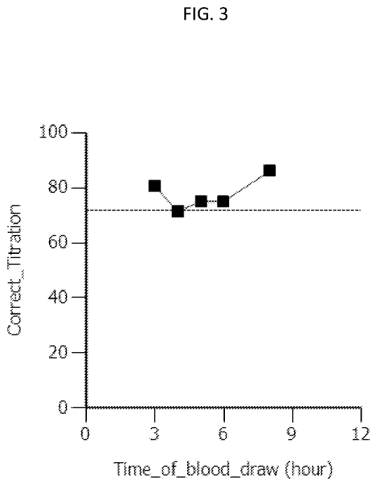

FIG. 3 suggests that dose titrations based on single blood draws 3-8 hr after morning dosing gave the best match with 24-hour T Cavg-based dose titration recommendations (72-86% correct titration decisions).

Table 1 summarizes the occurrence of each unnecessary titration.

TABLE 1
Potentially Incorrect Dose Decisions by Time
of Analysis of Plasma Sample (Day 7 and 14)

| Time (hr) | 3 | 4 | 5 | 6 | 8 |
|---|---|---|---|---|---|
| Cx < Cavg: Unnecessary up-titration | 11.1 | 0 | 2.8 | 0 | 13.9 |
| (%) | | | | | |
| Cx > Cavg: Unnecessary down-titration | 11.1 | 25 | 22.2 | 19.4 | 11.1 |
| (%) | | | | | |

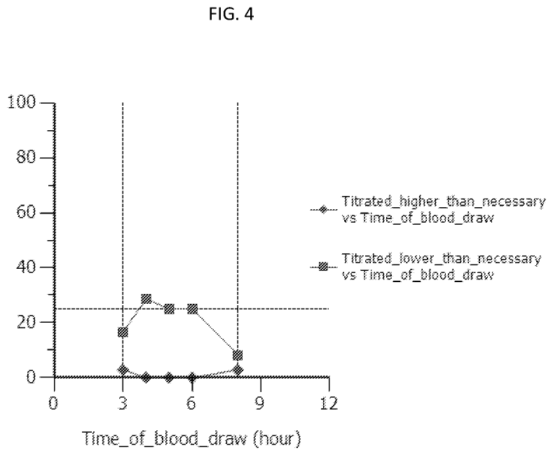

FIG. 4 illustrates the percentage of incorrect titration decisions based on a single blood draw resulting in doses that were higher or lower than necessary. As FIG. 4 suggests, it is reasonable to suggest that subjects should be titrated based on blood draws taken between 3 and 8 hr after morning dose of the drug.

Cmax-Based Decisions in Combination with Cavg

We also compared Cmax based decisions for the impact of different thresholds for down-titration decisions. Incorporation of Cmax into the titration algorithm addresses the safety risk of T levels greater than 1500 ng/dL.

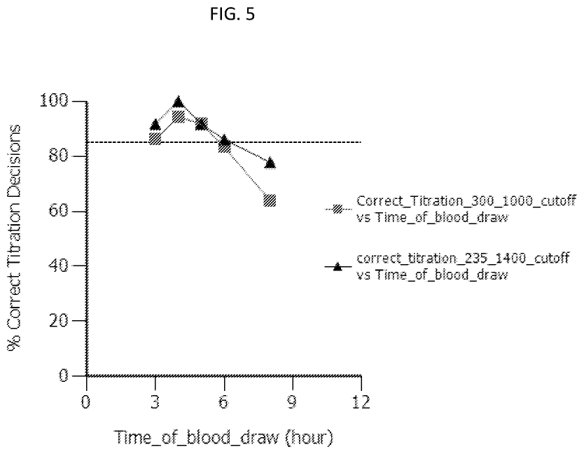

FIG. 5 Suggests that dose titrations based on single blood draws between 3 and 5 hr after morning dosing gives the best match with Cmax 0-12 based dose titration recommendations using the thresholds of 235 and 1400 ng/dL (91.7-100%). T-values (Cx) below the lower limit of 235 ng/dL results in up-titration to achieve Cavg within the normal range. T-values (Cx) above the upper limit of 1400 ng/dL result in down-titration to maintain Cmax values less than 1500 ng/dL. Application of the range 300-1000 ng/dL resulted in lower correct titration percentages. Additionally, 8 hrs post morning dosing gives a lower percentage of correct titration decisions, and thus is not recommended. For 6 hr post morning dosing, we observed 5 subjects in the Phase 2b study having Cmax over 1600 ng/dL but not meeting down-titration decisions based on plasma T level. Therefore, we propose to use the window of 3-5 hr for our Phase 3 trial.

TABLE 2
Potentially Incorrect Dose Decisions by Time of Analysis
of Plasma Sample (Day 7 and 14 data from Phase 2b study).

| | | | | | |
|---|---|---|---|---|---|
| Time (hr) | 3 | 4 | 5 | 6 | 8 |

Cut-off: 300-1000 ng/dL:

| | | | | | |
|---|---|---|---|---|---|
| Cx < Cavg: Unnecessary up-titration (%) | 11.1 | 0 | 2.8 | 8.3 | 27.8 |
| Cx > Cavg: Unnecessary down-titration | 2.8 | 5.6 | 5.6 | 8.3 | 8.3 |
| (%) | | | | | |

Cut-off: 235-1400 ng/dL:

| | | | | | |
|---|---|---|---|---|---|
| Cx < Cavg: Unnecessary up-titration (%) | 8.3 | 0 | 8.3 | 13.9 | 22.2 |
| Cx > Cavg: Unnecessary down-titration | 0 | 0 | 0 | 0 | 0 |
| (%) | | | | | |

Table 2 confirmed that 6 and 8 hr are not appropriate for single blood draw, and the window of 235-1400 ng/dL provided lower incorrect percentages for both cases.

It was proposed to use 3-5 hr post morning dose as the single blood draw time window. The up- and down-titration thresholds are set at 235 ng/dL and 1400 ng/dL, respectively to achieve a high percentage of correct decision while minimizing the percentage of incorrect decisions.

Both Days 49 and 84 in the Phase Ilb trial have 24-hour PK data. We evaluated data for these two days on 15 subjects who received 400 mg A.M./200 mg P.M. dose regimen, which is the starting dose in the Phase 3 trial. The proposed titration window of 3-5 hr single blood draw and 235-1400 ng/dL threshold values were validated by this approach.

Titration Scheme

In conclusion, we propose that the single blood draw used for titration decisions be obtained 3-5 hr post morning dose. Subjects having single blood draw values of total plasma T less than or equal to 235 ng/dL were up-titrated. Subjects having single blood draw values of total plasma T greater than 1120 ng/dL were down-titrated.

### Example 2. Adjustment of New Dosing Titration Scheme

Number of Subjects

Up to approximately 170 completed subjects consented to the Study 1 EXT Study to undergo 8 weeks or more of washout from study medication or interim testosterone replacement therapy, followed by a total of 180 days of treatment with SOV2012-F1. Subjects were dose-titrated to their final dose over the first 28-56 days of the treatment period. The Study 1 EXT Study included three to four 24-hour ABPM assessment sessions, depending on at which timepoint the subject entered the study (directly from Study 1 or as a Late Entry Subject to Study 1 EXT or as a newly enrolled Study 1—naïve subject).

Approximately 135 of the approximately 170 consented subjects (80%) were targeted to complete 120 days of the 180-day treatment period, including at minimum, the baseline Day 1 and the 4-month Day 120 required 24-hr ABPM assessment sessions in Study 1 EXT.

Treatment During Extension Study, Study 1 EXT

During the Study 1 EXT study period, all subjects were washed out from their originally assigned Study 1 study medication or any interim testosterone replacement for an 8-week period. At the completion of that washout, all subjects received SOV2012-F1, starting at a total daily dose of 400 mg (200 mg with the breakfast meal and 200 mg with the dinner meal) and were titrated, if needed, according to the dose titration algorithm established for the Study 1 EXT protocol. Dietary guidance and meal content were unchanged from Study 1 protocol.

Extension Study Duration
Primary Endpoint
   Change from baseline in 24-hour average ambulatory systolic blood pressure after approximately 120 days (±3) of treatment.
   To determine the response to a lower starting dose of oral SOV2012-F1 with up and down titration as appropriate, as measured by:
      Percentage of SOV2012-F1—treated subjects with a plasma T Cavg within the normal range after 90 days of treatment.
Secondary Endpoints
   Change from baseline in 24-hour average ambulatory systolic blood pressure after approximately 180 days (±3) of treatment.
   Change from baseline in 7 AM to 10:30 PM -hour average ambulatory systolic blood pressure (daytime) after approximately 120 days (±3) and 180 days (±3) of treatment.
   Change from baseline in 11 PM to 6:30 AM -hour average ambulatory systolic blood pressure (nighttime) after approximately 120 days (±3) and 180 days (±3) of treatment.
   Maximum 24-hour systolic blood pressure after approximately 120 days (±3) and 180 days (±3) of treatment.
   Change from baseline in 7 AM to 10:30 PM -hour average ambulatory diastolic blood pressure (daytime) after approximately 120 days (±3) and 180 days (±3) of treatment.
   Change from baseline in 11 PM to 6:30 AM -hour average ambulatory diastolic blood pressure (nighttime) after approximately 120 days (±3) and 180 days (±3) of treatment.
   Change from baseline in 24-hour mean diastolic blood pressure (dBP) measured by ABPM after 120 (±3) days and 180 (±3) days of treatment, in SOV2012-F1—treated subjects.
   Maximum 24-hour diastolic blood pressure after approximately 120 days (±3) and 180 days (±3) of treatment.
   Change from baseline in 24-hour average ambulatory heartrate after approximately 120 days (±3) and 180 days (±3) of treatment.
   Change from baseline in 7 AM to 10:30 PM -hour average ambulatory heartrate (daytime) after approximately 120 days (±3) and 180 days (±3) of treatment.
   Change from baseline in 11 PM to 6:30 AM -hour average ambulatory heartrate (nighttime) after approximately 120 days (±3) and 180 days (±3) of treatment.
   Observed and change from baseline in half hourly systolic blood pressure, diastolic blood pressure, and heartrate after approximately 120 days (±3) and 180 days (±3) of treatment.
   The percentage of SOV2012-F1—treated subjects with maximum plasma testosterone concentration (T Cmax) values after 90 days of treatment:
      <1500 ng/dL;
      >1800 to 2500 ng/dL;
      >2500 ng/dL.
Safety Endpoints
   To determine the incidence of AEs, SAEs, and AEs leading to Study 1 EXT withdrawal in SOV2012-F1-treated subjects.
   Observed and change from baseline in BP and HR obtained in-clinic during the treatment period.
   Observed and change from baseline in the following laboratory parameters in SOV2012-F1—treated subjects during the treatment period:
      Liver function tests (alanine aminotransferase [ALT], aspartate aminotransferase [AST], total bilirubin, alkaline phosphatase)
      Hematology parameters (hemoglobin)
      Hormone levels (luteinizing hormone [LH], follicle-stimulating hormone [FSH], DHT, sex hormone—binding globulin [SHBG], and thyroid-stimulating hormone [TSH])
      Lipid profiles (high-density lipoproteins, low-density lipoproteins, total cholesterol, and triglycerides)
      Serum PSA

SOV2012-F1 Dose Titration

The dose of study drug was titrated during the efficacy period using an algorithm that was developed using 90-day 24-hr PK data obtained from 133 Study 1 subjects in the SOV2012-F1 treatment group. Dose titration for each subject was based on the NaF/EDTA plasma T measured between 3 to 5 hours (+10min) after the morning dose at Day 14 and Day 42. Dose titrations occurred at Day 28 and Day 56 if needed, based on the following algorithm:
For subjects who may need dose titration at Day 28 based on the plasma T level obtained between 3 to 5 hours on Day 14:
   T3-5<400 ng/dL: dose increased to 600 mg (300 mg AM, 300 mg PM)
   T3-5≥400 to 900 ng/dL: no dose change
   T3-5>900 ng/dL: dose decreased to 200 mg (100 mg AM, 100 mg PM)
For subjects who may need dose titration at Day 56, based on the plasma T level obtained between 3 to 5 hours on Day 42:
   For subjects whose dose was not titrated previously (e.g., remained at 200 mg AM, 200 mg PM) and the resulting plasma T3-5 at Day 42 are:
      T3-5<400 ng/dL: dose increased to 600 mg (300 mg AM, 300 mg PM)
      T3-5≥400 to 900 ng/dL: no dose change
      T3-5>900 ng/dL: dose decreased to 200 mg (100 mg AM, 100 mg PM)
   For subjects whose dose was previously decreased to 200 mg (100 mg AM, 100 mg p.m.), and the resulting plasma T3-5 at Day 42 are:
      T3-5<400 ng/dL: dose increased to 400 mg (200 mg AM, 200 mg PM)
      T3-5≥400 to 900 ng/dL: no dose change
      T3-5>900 ng/dL: dose decreased to 100mg AM only.
   For subjects whose dose was previously increased to 600 mg (300 mg AM, 300 mg PM), and the resulting plasma T3-5 at Day 42 are:
      T3-5<400 ng/dL: dose increased to 800 mg (400 mg AM, 400 mg PM)
      T3-5≥400 to 900 ng/dL: no dose change
      T3-5>900 ng/dL: dose decreased to 400 mg (200 mg AM, 200 mg PM)

Measuring T at 3 to 6 hours

In the Study 1 EXT, it was identified that measuring plasma T concentration at from about 3 hours to about 6 hours after administration provided a reliable plasma concentration for the phytosterol ester-containing formulation administered with a meal. The T concentration over the period 0 to 6 hours post-dose for Visits on Days 14 and 42 of the Study 1 EXT. The T measurements were made using plasma samples collected with NaF/EDTA tubes and analyzed by LC-MS/MS. FIG. 7 shows a low T level from hours 0 to 2 post-dose and a relatively flat PK curve from hours 3 to 6 post-dose, illustrating the constant plasma T value in this window.

The ratio of plasma to serum results over the 3 to 6 hour window were also consistent. If the ratio changed significantly over the window, then the titration thresholds above would be time-dependent. Table 3 below shows that predetermined factor F varies within a narrow range, thereby allowing the adjustment of dose based on samples obtained in the 3 to 6 hour window.

TABLE 3
Predetermined factor F in post-dose window

| | Post-Dose Timepoint, hrs | | | |
|---|---|---|---|---|
| Plasma/Serum Ratio | 3 | 4 | 5 | 6 |
| Day 14 | 0.87 | 0.88 | 0.85 | 0.83 |
| Day 42 | 0.92 | 0.92 | 0.90 | 0.92 |
| Days 14 and 42 Mean over 3, 4 | 0.89 | | | — |
| and 5 hours | | | | |
| Days 14 and 42 Mean over 3, 4, 5 | 0.89 | | | |
| and 6 hours | | | | |

Efficacy (Cavg)

The primary efficacy endpoint was the percentage of SOV2012-F1—treated subjects with a 24-hour total T Cavg within the normal range after 90 days of treatment within the extension.

The Cavg was calculated as area under the concentration-time curve from time 0 to 24 hours (AUCO-24) divided by the actual number of hours between dosing and the 24-hour sample collection time.

Ambulatory Systolic Blood Pressure (sBP)

The change from baseline in the 24-hour average sBP was analyzed as the primary blood pressure endpoint. Key secondary analyses were derived from the changes from baseline in the daytime and night time sBP.

The difference in least squares means and associated 90% CI were provided.

Ambulatory Diastolic Blood Pressure (dBP) and Ambulatory Heartrate (HR)

These were evaluated in a similar fashion to the sBP except for the maximum heartrate. Hourly and half hourly observed and time matched change from baseline were descriptively summarized.

Cmax

The secondary endpoint was evaluated by estimating the proportion of SOV2012-F1 treated subjects Day 90 with T Cmax:

a) T Cmax≤1500 ng/dL

b) T Cmax>1800 and ≤2500 ng/dL

c) T Cmax>2500 ng/dL

Titration Decisions

Using the T concentrations obtained at the titration timepoints with EDTA and serum tubes, exploratory analysis was performed to compare the predicted titration decisions with those which were made using NaF-EDTA samples. Table 4 shows the dose distribution of subjects from the Study 1 and Study 1 EXT at Day 90.

TABLE 4
Non-final data at Day 90 of each study.

| Study: | 100 | 200 | 400 | 600 | 800 | 1000 |
|---|---|---|---|---|---|---|
| daily dose | mg | mg | mg | mg | mg | mg |
| Study 1 | Dose not | 3% | 28% | 61% | 7% | 1% |
| (n = 187) | used | | | | | |
| Study 1 EXT | 2% | 6% | 23% | 44% | 25% | Dose not |
| (n = 146) | | | | | | used |

In summary, there were subjects in Study 1 EXT who were started at 400 mg daily of SOV2012-F1 and had plasma T measurements obtained in the window of 3 to 5 hours post-morning dose, and who had no dose adjustments against titration thresholds of 400 and 900 ng/dl (plasma NAF/EDTA). These subjects had the surprising results of both Cavg in the normal range, Cmax values conforming to the FDA criteria, and blood pressure results of less than 3.8 mm increase (e.g. less than 2 mm increase, less than 3 mm increase). Heart rate increases were also superior (less increase versus baseline).

Additionally, we identified subjects who were started at 400 mg daily of SOV2012-F1, had plasma T measurements obtained in the window of 3 to 5 hours (or 3 to 6 hours) post-morning dose, and who had dose titrations against 400 and 900 ng/dL plasma (NaF/EDTA) thresholds (FIG. 6). These subjects had the surprising results of both Cavg in the normal range, Cmax values conforming to the FDA criteria, and blood pressure results of less than 4.9 mm increase (e.g. less than 2 mm increase, less than 3 mm increase). These blood pressure measurements were made by the ABPM protocol. Heart rate increases were also superior (less increase versus baseline).

### Example 3. Blood Pressure is Minimally Affected with this Titration Scheme

Ambulatory blood pressure measurements were obtained for 134 subjects as described above in Example 2. These data were directly compared to a TU formulation from Clarus Therapeutics that lacks phytosterol esters and was not subject to the same dosing titration regimen described herein. Table 5 below illustrates the results.

TABLE 5
Changes from baseline in blood pressure parameters in ABPM patients
in study CLAR-15012 (TU) and Study 1 EXT (SOV2012-F1)

| Increases | ALL ABPM patients | |
|---|---|---|
| Decreases | | SOV 2012-F1 |
| (LS means) | CLAR (N = 135) | (N = 134) |
| 24 hr heart rate HR* (bpm) | +2.2 | +0.71 |
| Daytime systolic BP (mmHg) | +5.0 | +1.70 |
| Nighttime systolic BP | +4.9 | +1.65 |
| (mmHg) | | |
| 24 hr average systolic BP (mmHg) | +4.9 | +1.64 |

*24 hour average heart rate in beats per minute, LS = least squared means

These data show that ABPM measurements for all patients increased by only 1.70 mmHg during the daytime, 1.65 mmHg during the nighttime, and 1.64 mmHg over a 24-hour average, relative to baseline, as compared to 5.0 mmHg, 4.9 mmHg, and 4.9 mmHg, respectively, for patients undergoing treatment with the Clarus formulation.

Furthermore, in-clinic systolic blood pressure and heart rate data for the Study 1 and Study 1 EXT are provided below. As is shown in Tables 6 and 7 below, the Study 1 EXT produced a slower rise and lower maximum systolic blood pressure measurements than the Study 1 protocol. The average change from baseline for heartrate from the 90^{th} day to the 180^{th} day for the Study 1 EXT was 2.2 beats per minute (bpm), and the average change of the Study 1 (Days 90 and 180) was 3.3 bpm change from baseline.

TABLE 6
Study 1 and Study 1 EXT Heart Rate Data

| | Study 1 | Study 1 EXT |
|---|---|---|
| | SOV2012-F1 | SOV2012-F1 |
| | (N = 214) | (N = 106) |
| Baseline | | |
| n | 214 | 104 |
| Mean(SE) | 69.93 (0.573) | 70.95 (0.927) |
| 95 CI | 68.80, 71.06 | 69.11, 72.79 |
| Day 14 | | |
| n | 205 | 99 |
| Mean(SE) | 2.71 (0.518) | 0.93 (0.784) |
| 95 CI | 1.69, 3.74 | −0.63, 2.49 |
| Day 42 | | |
| n | 197 | 98 |
| Mean(SE) | 3.84 (0.641) | 1.69 (0.939) |
| 95 CI | 2.57, 5.10 | −0.17, 3.56 |
| Day 90 | | |
| n | 187 | 98 |
| Mean(SE) | 4.34 (0.610) | 2.12 (0.920) |
| 95 CI | 3.14, 5.55 | 0.30, 3.95 |
| Day 119 | NA | |
| n | NA | 93 |
| Mean(SE) | NA | 1.24 (0.919) |
| 95 CI | NA | −0.59, 3.06 |
| Day 180 | | |
| n | 176 | 64 |
| Mean(SE) | 2.26 (0.641) | 3.14 (1.253) |
| 95 CI | 1.00, 3.53 | 0.64, 5.64 |

TABLE 7
Study 1 and Study 1 EXT Systolic Blood Pressure Data

| | Study 1 | Study 1 EXT |
|---|---|---|
| | SOV2012-F1 | SOV2012-F1 |
| | (N = 214) | (N = 106) |
| Baseline | | |
| n | 214 | 104 |
| Mean(SE) | 125.99 (0.618) | 125.64 (0.986) |
| 95 CI | 124.77, 127.21 | 123.69, 127.60 |
| Day 14 | | |
| n | 205 | 99 |
| Mean(SE) | 0.92 (0.628) | 1.07 (1.030) |
| 95 CI | −0.31, 2.16 | −0.97, 3.12 |
| Day 42 | | |
| n | 197 | 98 |
| Mean(SE) | 2.59 (0.749) | 1.05 (0.946) |
| 95 CI | 1.11, 4.06 | −0.83, 2.93 |
| Day 90 | | |
| n | 187 | 98 |
| Mean(SE) | 2.05 (0.756) | 2.30 (1.039) |
| 95 CI | 0.56, 3.54 | 0.23, 4.36 |
| Day 119 | NA | |
| n | NA | 93 |
| Mean(SE) | NA | 3.44 (1.035) |
| 95 CI | NA | 1.39, 5.50 |
| Day 180 | | |
| n | 176 | 64 |
| Mean(SE) | 2.19 (0.795) | 3.23 (1.091) |
| 95 CI | 0.62, 3.76 | 1.06, 5.41 |

### Example 4. Effects of an Oral TU formulation (SOV2012-F1) on Ambulatory Blood Pressure in Hypogonadal Men

### Methods

The study was an open-label, multicenter, single arm study with an untreated screening period at baseline visit to assess BP and heart rate via 24-hour ambulatory BP monitoring (ABPM) prior to administration of study medication, and two visits at 120 days and 180 days after initiating oral testosterone undecanoate. In addition, seated clinic BP measurements were performed at all study visits. All study participants initially received oral testosterone undecanoate at a dose of 200 mg twice daily with breakfast and dinner meals. Based on thresholds of morning plasma testosterone between 3-5 hours post morning dose (<400 ng/dL to titrate upwards or >900 ng/dL to titrate downwards), dose decreases to 100 mg twice daily or increases to 300 mg twice daily) took place at day 28. A further potential titration of dose (decrease to a minimum of 100 mg daily or increase to a maximum of 400 mg twice daily) occurred at day 56 to achieve therapeutic levels of plasma testosterone; the day 56 dose was maintained until end of treatment (withdrawal or 180 days).

### Study Participants

All participants were men between 18 and 65 years of age, inclusive, with documented hypogonadism as defined by a below normal serum testosterone and at least one sign or symptom of testosterone deficiency. The total serum testosterone level was required to be ≤281 ng/dL on 2 consecutive blood samples obtained between 7 and 10 am on separate days, at least 3 days apart either in individuals naïve to androgen replacement or following at least 8 weeks of washout of current androgen therapy (washout periods up to 6 months were required for testosterone implants). Also required was that there was no change in medications, including antihypertensive agents, within the 3 months prior to enrollment and that the mean clinic BP was ≤140 systolic and ≤90 diastolic. Subjects with uncontrolled hypertension (clinic BP>140/90 mmHg) were excluded based on FDA guidance. The main exclusion criteria were the use of any medications or clinical conditions that could affect absorption or levels of testosterone undecanoate; hemoglobin A1c>8%; hemoglobin<11.0 g/dL or >16.0 g/dL; serum transaminases>2 times the upper limits of normal; estimated glomerular filtration rate of <60 ml/min/1.73 m, or prostatic specific antigen (PSA)>2.5 ng/ml and/or an abnormal prostate gland on palpation. Additionally, exclusionary criteria due to the ambulatory BP monitoring procedures were an upper-arm circumference>45 cm; long-distance driving or a planned trip of >60 minutes while wearing the monitor and cardiac arrhythmias (e.g., atrial fibrillation) that might interfere with the ability of the ambulatory BP recorder to obtain reliable measurements.

The trial was conducted in accordance with Good Clinical Practice requirements, as described in the current revision of International Conference on Harmonization of Technical Requirements of Pharmaceuticals for Human Use (ICH) guidelines and the Declaration of Helsinki. The study protocol and informed consent forms were reviewed and approved by the Copernicus Group Institutional Review Board (Cary, NC, USA). Before any study procedures could occur, a written informed consent was obtained from each study participant.

### Safety Assessments

Clinical evaluation and vital signs were assessed at baseline and after 14, 42, 90, 119 and 179 days. At each clinic visit following the screening visit, all study participants were queried about adverse events and a symptom-directed physical examination was performed as indicated clinically. Laboratory tests were assessed at baseline and after 90 and 180 days.

### Blood Pressure Monitoring

Blood pressure was monitored manually in the clinic at the baseline and post-treatment study visits. The clinic measurements were made in the seated position in triplicate after 10 minutes of rest and using appropriately sized cuff and bladder with a digital recorder. Any study participant with a baseline clinic average BP>140/90 mmHg was withdrawn from participation in the trial. For ambulatory BP measurements, study participants were fitted with a recorder that was initiated to measure the BP at 30-minute intervals during the day (7:00 am to 11:00 pm) and night (11:00 pm to 7:00 am) (Spacelabs Medical Model 90207; Redmond, WA). The ABPM data were evaluated both manually and programmatically by standardized, computerized methods, for validity and required that no more than 4 consecutive timepoints were missing, no more than 10 of the possible 48 timepoints over 24 hours were missing, and at least 22 of 24 hours had valid data. If these quality control criteria were not met, the study could be repeated within 48 hours of the failed ambulatory BP procedure.

### Statistical Analyses

The 24-hour, daytime and nighttime average systolic and diastolic BPs were summarized with means and 95% confidence intervals (CIs), and cumulative distribution curves. Using a mixed model repeated measures (MMRM) analysis with study participant as a random effect (all participants with non-missing post-baseline results), and visit, baseline diabetes status and baseline antihypertensive treatment status as fixed effects; direct comparisons of visits were performed. The least squares mean at each visit and the least squares mean for the difference between 120 days and baseline with the associated 95% CIs were calculated. Cumulative distribution function curves of change from baseline to Day 120 and Day 180 were also performed. The primary endpoint in this BP safety study was the change from baseline to day 120 for the average 24-hour systolic BP. A key secondary endpoint was the change from baseline to day 180 for the average 24-hour systolic BP. Comparisons were also made for the ambulatory BP changes at 180 versus 120 days. Other assessments included changes from baseline in the awake (daytime) and sleep (nighttime) systolic BP, the 24-hour, awake, and sleep diastolic BPs and the 24-hour, awake and sleep heart rates. Additionally, the BP and heart rate changes were evaluated in subgroups of study participants with and without antihypertensive therapy at baseline and with and without a baseline history of diabetes mellitus. The incidence of adverse events was tabulated in all participants who received at least one dose of study drug (safety population).

The change of 24-hour BP from baseline was calculated using the time-weighted average BP obtained over 24 hours divided by the time duration. Changes in hourly average BPs were calculated by taking the difference between the corresponding hourly BP at the end of the treatment visits and the baseline visit for a given post-dosing hour. Post-hoc analyses were also performed to assess relationships among changes from baseline in ambulatory systolic and diastolic BP with changes in body weight, heart rate, testosterone concentration and hemoglobin.

### Sample Size Calculation.

A sample size of 135 subjects would yield a two-sided 90% confidence interval with a distance from the difference in means to the limits that was equal to 1.4 mm Hg when the estimated standard deviation of the differences for 120 days versus baseline for the 24-hour mean systolic BP was 10 mmHg. In addition, a sample size of 119 study participants achieved 90% power to detect non-inferiority (versus baseline) using a one-sided one-sample t-test when the non-inferiority margin was 3.0 mmHg, the actual mean was 0, and the significance level (α) of the test was 0.025. Assuming a 10% drop-out or non-evaluable ambulatory BP monitoring rate, 133 study participants would be required for enrollment to achieve 119 evaluable study participants.

### RESULTS

### Subject Disposition and Baseline Characteristics

A total of 155 study participants were enrolled and received at least 1 dose of study drug. Of these 155 participants, 153 also had an evaluable ambulatory BP study at baseline and two were discontinued from further participation in the study. One hundred thirty-six (89%) completed the 120-day visit and 125 (82%) completed the 180-day visit and had valid baseline and successful on-treatment ABPM studies. The primary reasons for early termination were withdrawal by the subject (5.6%), adverse events (1.3%), lost to follow-up (6%) and other (2.6%). The demographic and baseline characteristics of the study participants are shown in Table 8. The mean age at baseline was 51.2 years (52% older than 50 years), 77% were white and 19% were black. Thirty-seven percent (56 of 155) of the study participants were taking antihypertensive therapy. There were no dose increases in antihypertensive medications however, 5 (3.2%) were started on new antihypertensive agents during the 180-day study. Twenty-two percent (34 of 155) of the study cohort had a history of diabetes mellitus. A greater percentage of study participants taking antihypertensive therapy had diabetes (44.6%) than those not taking antihypertensive therapy (9.1%). A high percentage (96%) of study participants with both diabetes and who were taking antihypertensive therapy were obese (body mass index≥30 kg/m2) whereas for those participants without these comorbidities, 56% were obese. The percentage of study participants achieving a normal testosterone (plasma collected in NaF/EDTA tubes) after 90 days of treatment was 96.1% (plasma C_{avg0-24}=393.5 ng/dL); quantitation was by liquid chromatography-mass spectrometry (3, 4). A study in 105 healthy eugonadal subjects found a normal range for total testosterone to be 222 to 800 ng/dL when using the NaF/EDTA plasma sample collection tube.

TABLE 8
Characteristics of the Patient Population at Baseline (n = 155)

| | |
|---|---|
| Characteristic | Value* |

Age (years)

| | | |
|---|---|---|
| Mean | 51.2 | (9.4) |

Race, N (%)

| | | |
|---|---|---|
| Asian | 4 | (2.6) |
| Black | 29 | (18.7) |
| White | 119 | (76.8) |
| Other | 3 | (1.9) |

Body mass index (BMI) (kg/m²)

| | | |
|---|---|---|
| Mean (SD) | 34.0 | (7.3) |

Medical history, N (%)

| | | |
|---|---|---|
| On antihypertensive therapy | 56 | (36.1) |
| Type 2 diabetes | 34 | (21.9) |
| On statin therapy | 38 | (24.5) |
| Cardiovascular disease | 17 | (12.3) |

Blood Pressure (mmHg)
Clinic, mean (SD)

| | | |
|---|---|---|
| Systolic | 126.1 | (9.8) |
| Diastolic | 78.7 | (6.7) |

Ambulatory, mean (95% CI)

| | | |
|---|---|---|
| 24-hour Systolic | 128.9 | (126.8, 131.0)) |
| 24-hour Diastolic | 76.2 | (74.6, 77.9) |
| Daytime Systolic | 132.7 | (130.5, 134.8) |
| Daytime Diastolic | 79.2 | (77.5, 80.9) |
| Nighttime Systolic | 121.0 | (118.7, 123.3) |
| Nighttime Diastolic | 70.0 | (68.2, 71.8) |

Heart rate

| | | |
|---|---|---|
| Clinic, mean (SD) | 71.9 | (9.5) |
| 24-hour, mean (95% CI) | 76.3 | (74.4, 78.3) |

### Blood Pressure and Heart Rate

Change from baseline in the 24-hour ambulatory systolic BP on oral testosterone undecanoate following 120 days of treatment was 1.7 mmHg, p=0.018 (Table 9). Lesser effects were seen for the ambulatory diastolic BP and were not statistically significant (Table 9). Results following 180 days of oral testosterone undecanoate therapy were comparable to the 120 day results (Table 9). The nocturnal decline in systolic BP (daytime-nighttime/daytime BP×100 (%)) was unchanged by the oral testosterone undecanoate therapy (8.8% at baseline versus 8.9%) at day 120 of the study. Small increases in the 24-hour ambulatory heart rate were observed following 120 and 180 days of therapy (0.7 and 1.9 beats/minute, respectively) (Table 9).

TABLE 9
Changes from Baseline in Blood Pressure and Heart
Rate Following Oral Testosterone Undecanoate

| | | | |
|---|---|---|---|
| | | Change from | |
| Parameter | Mean (SD) | baseline, (95% CI) | P-value ¹ |

Clinic Systolic BP (mmHg)

| | | | |
|---|---|---|---|
| Baseline (n = 152) | 126.1 (9.8) | | |
| Day 120 (n = 141) | 128.5 (10.9) | 2.7 (0.9, 4.5) | 0.003 |
| Day 180 (n = 132) | 128.1 (10.0) | 2.4 (0.6, 4.2) | 0.010 |

Clinic Diastolic BP (mmHg)

| | | | |
|---|---|---|---|
| Baseline | 78.7 (6.7) | | |
| Day 120 | 80.0 (7.7) | 1.5 (0.3, 2.6) | 0.017 |
| Day 180 | 80.1 (7.6) | 1.7 (0.5, 2.9) | 0.006 |

Clinic heart Rate (beats/min)

| | | | |
|---|---|---|---|
| Baseline | 71.9 (9.5) | | |
| Day 120 | 72.9 (9.4) | 1.1 (−0.4, 2.6) | 0.152 |
| Day 180 | 74.3 (10.3) | 2.6 (1.0, 4.2) | 0.002 |

| | | | |
|---|---|---|---|
| | | Change from | |
| Parameter | Mean (SE) | baseline (95% CI)* | P-value ² |

24-hour ambulatory systolic BP (mmHg)

| Baseline (n = 153) | 128.9 | (1.1) | | | |
|---|---|---|---|---|---|
| Day 120 (n = 136) | 130.6 | (1.1) | 1.7 | (0.3, 3.1) | 0.018 |
| Day 180 (n = 125) | 130.7 | (1.1) | 1.8 | (0.3, 3.2) | 0.016 |

24-hour ambulatory diastolic BP (mmHg)

| Baseline | 76.2 | (0.8) | | | |
|---|---|---|---|---|---|
| Day 120 | 76.9 | (0.8) | 0.6 | (−0.3, 1.6) | 0.193 |
| Day 180 | 76.9 | (0.8) | 0.6 | (−0.4, 1.6) | 0.210 |

24-hour ambulatory heart rate (beats/minute)

| | | | | | |
|---|---|---|---|---|---|
| Baseline | 76.3 | (1.0) | | | |
| Day 120 | 77.0 | (1.0) | 0.7 | (−0.5, 1.9) | 0.261 |
| Day 180 | 78.2 | (1.0) | 1.9 | (0.6, 3.1) | 0.004 |

*Clinic BPs were assessed prior to the start of corresponding ambulatory BP assessment.
¹ P-values based on paired t test.
² Based on mixed model repeated measures analysis with visit, prior randomized treatment, baseline antihypertensive treatment status, and baseline diabetes status as fixed effects and subject as a random effect.
*Least square mean changes for ambulatory BP and heart rate

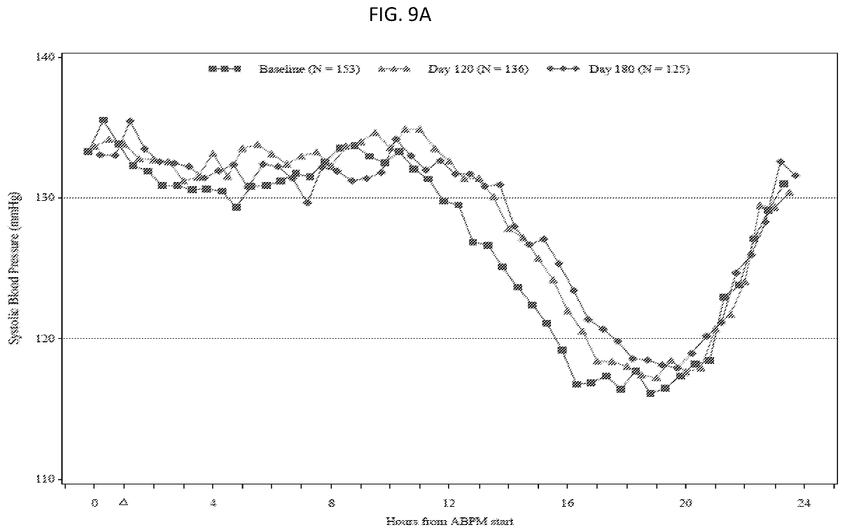

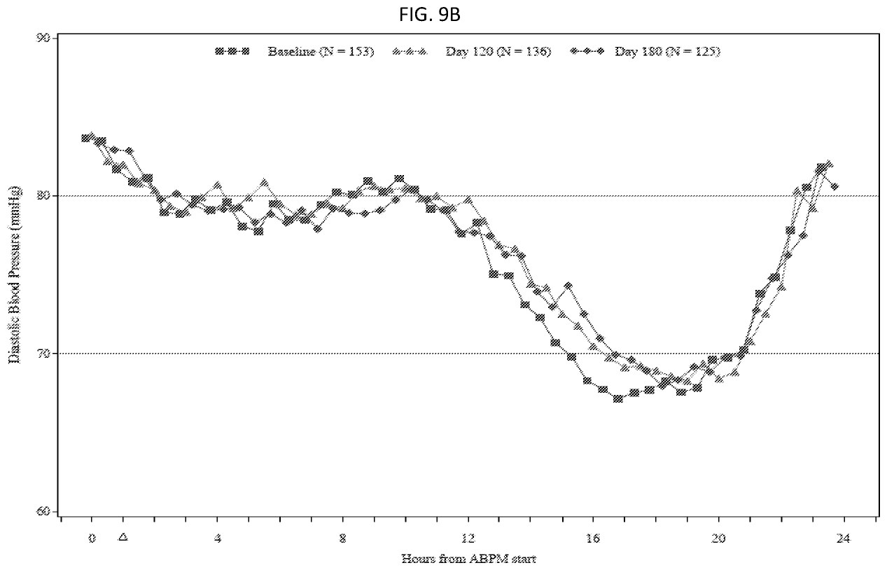

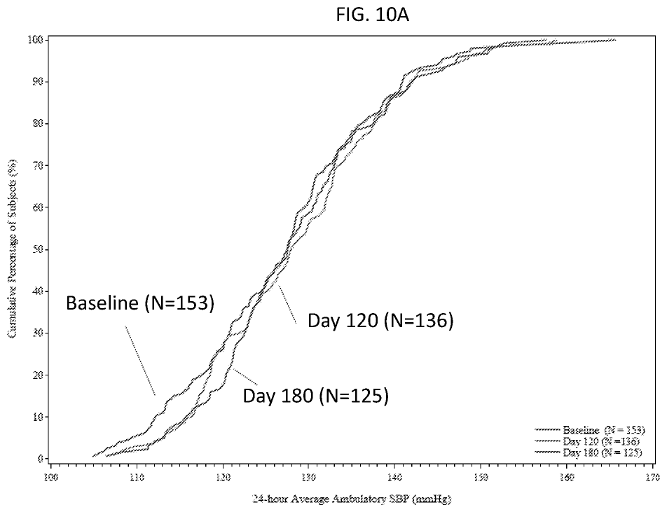

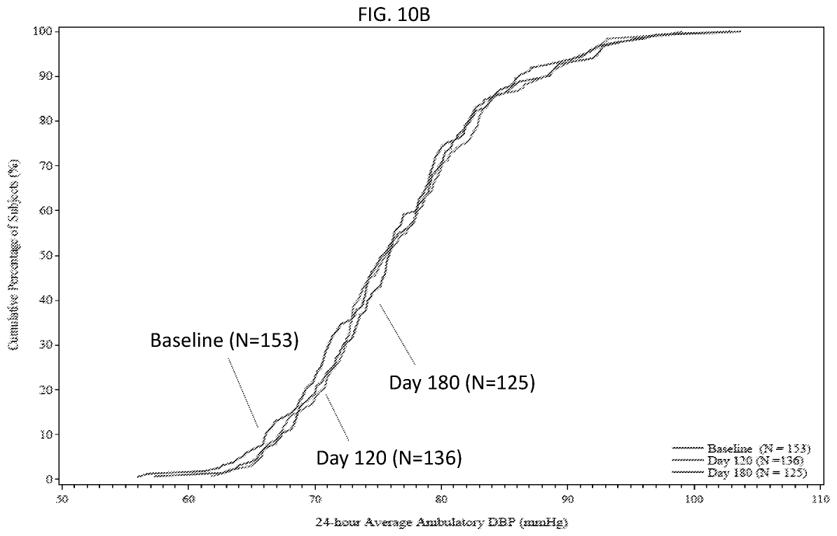

Ambulatory systolic and diastolic BPs over 24 hours at baseline and at the end of the 120- and 180-day treatment periods are shown in FIGS. 9A and 9B. The BP over 24-hours was higher following 120 and 180 days of treatment with oral testosterone undecanoate primarily between the hours 13 to 16 after initiation of the ambulatory BP monitoring. The effects on diastolic BP over 24-hours were less than for the systolic BP, particularly toward the end of the dosing periods. Cumulative distribution function (CDF) curves for the 24-hour ambulatory systolic and diastolic BPs are shown in FIGS. 10A and 10B. For systolic BP, there was separation of the CDF curves at days 120 and 180 versus the baseline period observed primarily when the 24-hour systolic BP values were <125 mmHg. Changes in 24 hour diastolic BP were negligible (FIGS. 10A and 10B).

The clinic blood pressure increased by 2.7/1.5 mmHg following 120 days of treatment with oral testosterone undecanoate and 1.7/1.7 mmHg following 180 days of treatment with oral testosterone undecanoate (Table 9). The clinic pulse rate increased by 1.1 and 2.6 beats/minute, respectively at days 120 and 180 (Table 9).

### Blood Pressure Changes in Subgroups on Antihypertensive Therapy or With Type 2 Diabetes

Changes from baseline in 24-hour blood pressure and heart rate at 120 days in study participants with and without antihypertensive therapy and with and without type 2 diabetes are shown in Table 10. Changes from baseline in 24-hour systolic and diastolic BP and heart rate were greater in patients taking antihypertensive drugs versus those without antihypertensive therapy.

TABLE 10
Changes from baseline at Day 120 in 24-hour blood pressure and heart rate in
study participants with and without antihypertensive therapy and with and
without diabetes mellitus following treatment with oral testosterone undecanoate

| | Systolic BP (mmHg) | | Diastolic BP (mmHg) | | Heart Rate (beats/minute) | |
|---|---|---|---|---|---|---|
| | | Change | | Change | | Change |
| | | from | | from | | from |
| | | baseline at | | baseline at | | baseline at |
| | | day 120 | | day 120 | | day 120 |
| Subgroup | Baseline | (95% CI) | Baseline | (95% CI) | Baseline | (95% CI) |
| With anti- | 131.3 | 3.4 | 75.9 | 1.8 | 75.8 | 1.3 |
| hypertensive | (127.8, 134.8) | (1.0, 5.9)** | (73.3, 78.6) | (0.2, 3.5)* | (72.6, 79.3) | (−0.9, 3.5) |
| therapy (n = 49) | | | | | | |
| Without | 127.9 | 0.7 | 78.2 | 0.0 | 76.9 | 0.4 |
| antihypertensive | (124.9, 130.9) | (−1.0, 2.4) | (75.8, 80.5) | (−1.2, 1.2) | (73.9, 79.8) | (−1.1, 1.9) |
| therapy | | | | | | |
| (n = 90) | | | | | | |
| With diabetes | 130.8 | 3.0 | 76.3 | 1.7 | 77.7 | 1.9 |
| mellitus | (125.9, 135.6) | (−0.2, 6.2) | (73.1, 79.6) | (−0.3, 3.7) | (73.7, 81.7) | (−1.1, 4.9) |
| (n = 29) | | | | | | |
| Without diabetes | 127.9 | 1.3 | 76.9 | 0.4 | 74.5 | 0.4 |
| mellitus | (125.8, 130.1) | (−0.2, 2.9) | (75.2, 78.6) | (−0.8, 1.5) | (72.4, 76.6) | (−1.0, 1.7) |
| (n = 110) | | | | | | |

Values are least square mean based on a mixed model repeated measures analysis with visit, prior treatment, baseline antihypertensive treatment status or baseline diabetes status as fixed effects and subject as a random effect.
*p < 0.05;
**p < 0.01

There were 33 study participants with diabetes mellitus and 120 study participants without diabetes mellitus at baseline who had evaluable ambulatory BP data. Changes from baseline in 24-hour systolic and diastolic BP at Day 120 were numerically, but not significantly greater in patients with type 2 diabetes versus those without type 2 diabetes. Similarly, changes in ambulatory heart rate were nominally greater in patients with diabetes versus those without diabetes.

Of note, for the study participants with type 2 diabetes, 25 (74%) were on antihypertensive therapy, thus there is substantial overlap of diabetic and antihypertensive therapy participants. There were 89 subjects in the study without antihypertensive medications at baseline, of whom only 9 had diabetes mellitus. For these 89 subjects, the 95% confidence interval for the change from baseline in 24-hour systolic and diastolic BP at 120 days was 0.8 mmHg and not statistically significant (data not shown).

### Blood Pressure Findings as a Function of Clinical Characteristics

Changes in the primary endpoint as a function of baseline ambulatory systolic BP, age, dose of oral testosterone undecanoate, body weight, and antihypertensive treatment status are shown in Table 11. At both days 120 and 180, baseline blood pressure and hypertension treatment status were significantly related to the changes in 24-hour systolic BP. Other clinical characteristics had no significant relationship with the primary endpoint at either of days 120 and 180.

TABLE 11
Change in 24-hour Average Ambulatory SBP as a Function
of Baseline SBP, Dose, Age, Weight, Diabetes and
Baseline Hypertensive Treatment Status by Visit

| | | | |
|---|---|---|---|
| Visit | | | |
| Covariate | Estimate | 95% CI | p-value |

Day 120

| | | | |
|---|---|---|---|
| Intercept | 49.523 | (32.352, 66.694) | <.0001 |
| Systolic Blood Pressure at | −0.433 | (−0.556, −0.310) | <.0001 |
| Baseline | | | |
| Age | 0.053 | (−0.100, 0.207) | 0.493 |
| Weight at Baseline | 0.005 | (−0.059, 0.068) | 0.889 |
| Dose (600 mg) | 3.020 | (−0.123, 6.163) | 0.060 |
| Dose (800 mg) | 4.201 | (0.469, 7.934) | 0.028 |
| Diabetic Status (With Diabetes | 0.387 | (−3.280, 4.054) | 0.835 |
| Mellitus) | | | |
| Hypertension Treatment Status | 4.330 | (1.230, 7.431) | 0.007 |
| (With Antihypertensive Therapy | | | |
| at Baseline) | | | |

Day 180

| | | | |
|---|---|---|---|
| Intercept | 48.474 | (33.205, 63.742) | <.0001 |
| Systolic Blood Pressure at | −0.402 | (−0.510, −0.295) | <.0001 |
| Baseline | | | |
| Age | 0.021 | (−0.118, 0.160) | 0.765 |
| Weight at Baseline | 0.007 | (−0.048, 0.062) | 0.809 |
| Dose (600 mg) | 1.091 | (−1.697, 3.880) | 0.440 |
| Dose (800 mg) | 2.962 | (−0.539, 6.464) | 0.097 |
| Diabetic Status (With Diabetes | 1.798 | (−1.478, 5.075) | 0.279 |
| Mellitus) | | | |
| Hypertension Treatment Status | 2.905 | (0.102, 5.708) | 0.042 |
| (With Antihypertensive Therapy | | | |
| at Baseline) | | | |

Intercept includes the effect of doses below 600 mg.

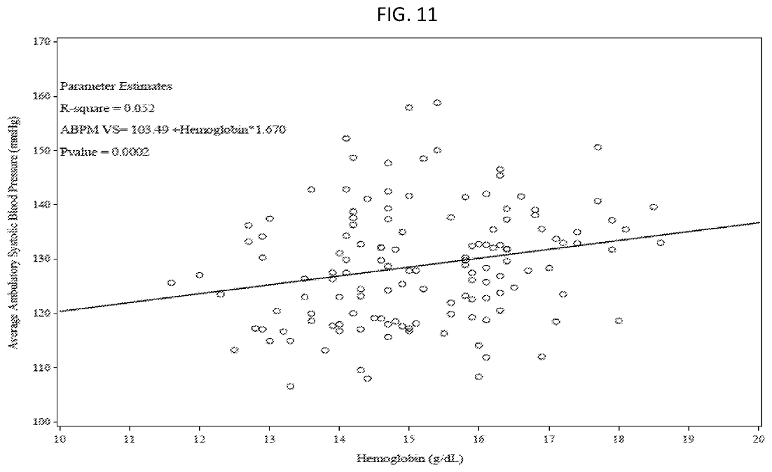

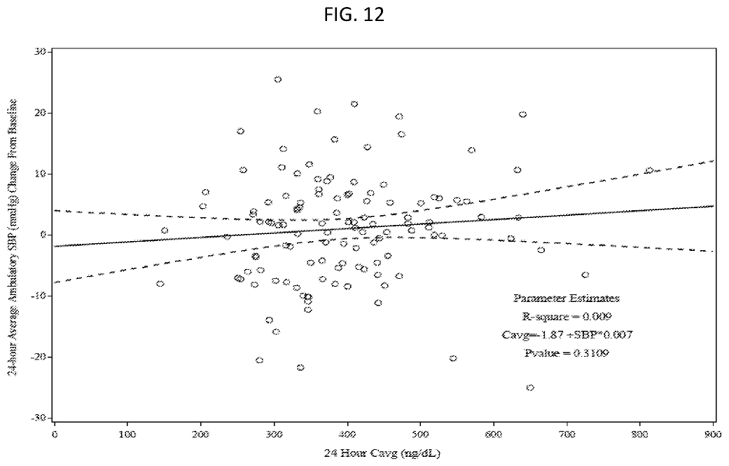

The serum hemoglobin was 14.7±1.1 g/dL at baseline, 15.1±1.5 g/dL at day 90 and 15.2±1.5 g/dL at day 180 (hemoglobin values were not obtained at day 120). The levels of hemoglobin at day 90 of the study had a weak, but significant relationship with 24-hour ambulatory systolic BP at days 120 and 180 (R²=0.052, p=0.0002 for day 120 and R²=0.049, p=0.0005 for day 180) (FIG. 11). However, the change from baseline in serum hemoglobin was not a predictor of changes in 24-hour ambulatory systolic BP in the study. Regression analyses of change from baseline in 24-hour systolic BP after 120 days versus 24-hour testosterone average concentration (after 90 days) showed no relationship (R²=0.009, p=0.3109), (FIG. 12). The testosterone undecanoate dose was constant in this study after 56 days until the end of treatment, nominally 180 days.

### DISCUSSION

The results of our blood pressure safety study demonstrated that the oral testosterone undecanoate formulation SOV2012-F1 was associated with small increases in clinic and ambulatory systolic BP following approximately 120 and 180 days of replacement therapy in hypogonadal men. No differences were observed between visits at 120 and 180 days suggesting that the impact of the drug on BP had reached a plateau by 120 days. The increases were larger for the systolic BP than for the diastolic BP, both when measured in the clinical setting as well as with 24-hour ambulatory BP monitoring. There were also small increases in the clinic and ambulatory heart rates observed on this oral testosterone undecanoate. The increases in ambulatory systolic BP were inversely related to baseline levels of ambulatory BP (likely related in part to regression to the mean) as well as antihypertensive treatment status but were not related to ambulatory heart rate, body weight, diabetes mellitus or changes in hemoglobin or testosterone levels. These findings are meaningful since a relation to heart rate might have suggested increases in sympathetic nervous system activity and relations with changes in body weight or hemoglobin in men treated with oral testosterone undecanoate might have supported an increase in plasma volume as one possible mechanism for the small increases in BP.

The study was designed to evaluate changes in the 24-hour ambulatory BP as the primary outcome measure in this study rather than changes in the clinic BP. Of note, the US Food and Drug Administration has advocated for use of ambulatory BP measurements in drug safety research since these devices have the potential to detect smaller changes in BP with improved reproducibility compared to clinical BP measurements and have virtually no placebo effects. As a result of the objectivity of ambulatory BP measurements, a placebo treatment arm for studies such as ours are not a requirement to provide evidence for a modest-to-moderate BP effect. The frequent readings obtained over a 24-hour period improves the precision of a BP safety study with statistical power to exclude 3 to 4 mmHg shifts in BP. It is also noteworthy that changes in clinic BP were slightly greater than the changes in 24-hour ambulatory BP in this study. This phenomenon is not uncommon and may be associated with a ‘white-coat’ effect seen with clinical readings that are abolished by ambulatory BP measurements.

We observed larger changes from baseline in ambulatory BP in those men taking antihypertensive drug therapy and in those with type 2 diabetes. The average change in ambulatory BP in those men on antihypertensive therapy was about 3.4/1.8 mmHg whereas those not taking antihypertensive therapy had a substantially smaller and insignificant change of 0.7/0.0 mmHg. Similar findings were observed for those patients with and without diabetes although a high proportion of the study participants with diabetes were also taking antihypertensive therapy and are not truly separate sub-populations.

The increases in 24-hour ambulatory BP on treatment in our study were modest (SBP 1.7 mmHg, with 95% CI 0.3, 3.1) and lower than with previous testosterone studies that employed both clinic and ambulatory BP measurements. These previous studies used testosterone undecanoate (oral) and testosterone enanthate (subcutaneous) routes of administration and reported increases in ambulatory SBP of 4.9 and 3.7) mmHg, respectively. For the previously reported study of oral TU, the increase in ambulatory systolic BP for subjects receiving antihypertensive medication was 5.5 mmHg. The mechanism for the lesser increase in BP observed with SOV2012-F1 is not entirely known may be due in part to the presence of phytosterol esters in the formulation, which have been associated with reductions in BP.

Increases in clinic systolic BP of 3 to 5 mmHg in prospective studies of large populations have been shown to have strong relationships with adverse cardiovascular events, particularly heart failure and stroke. However, the clinical importance of the small increases in BP in hypogonadal men observed in our study is less clear since men with testosterone deficiencies have increases in cardiovascular risk and there are data that suggest that normalizing testosterone levels in hypogonadal men may be associated with lower rates of cardiovascular morbidity than men who stay at low testosterone levels. Nevertheless, in hypogonadal men who require testosterone replacement, particularly those with a history of hypertension, careful clinical assessment for possible increases in BP remains important in clinical practice.

### CONCLUSIONS

In conclusion, this new oral formulation of testosterone undecanoate dosed between 100 mg once daily and 400 mg twice daily induced small increases in clinic and ambulatory BP. There were minimal increases in ambulatory heart rate that were not related to ambulatory BP changes. Study participants with a history of hypertension taking antihypertensive therapy and those with type 2 diabetes had larger increases in both ambulatory BP and heart rate following chronic oral testosterone undecanoate therapy than those without these 2 comorbidities. Hypogonadal men who were not receiving antihypertensive medication had negligible changes in ambulatory BP and heart rate.

### Example 5. Cmax and Cavg Measurements

Oral TU formulation formulated as SOV-2012-F1 was administered to male subjects in need of testosterone replacement therapy,
1. the resulting population averages have a Cmax/Cavg ratio for 0-24 hours of less than 2.5;
2. the resulting population averages have a Cmax/Cavg ratio for 0-12 hours of less than 2.2;
3. the resulting population averages have a Cmax/Cavg ratio for 12-24 hours of less than 2.2 when BID dosing is used;
4. the dose normalized dose is about 0.7×10^{-6/}dL (393.3 ng/dL/571 mg avg dose)
5. the daily dose range is 100 mg to 800 mg TU/day; daily doses of 200 mg or more may either be administered as a single dose or split 50:50 to be administered twice per day.
6. Systolic blood pressure, when assessed by Ambulatory Blood Pressure Monitoring (ABPM), after 4 months shows a mean increase of 1.7 mm Hg, and after 6 months a mean increase of 1.8 mm Hg.

### Advantages of Lower Ratios

Lower ratios have several advantages in a QD or BID context, including:

More reliable titration: Given the shape of the adsorption and elimination portions of the pharmacokinetic curve, it is desirable to choose a time or range of times for sampling the testosterone concentration that is both convenient for the individual and healthcare provider and carries a low degree of variability. For example, if the Cmax/Cavg ratio is high, implying a steep elimination portion of the PK curve, then variability is introduced into the T concentration of the sample taken for titration or periodic evaluation purposes because of factors such as: fat content of meal which affects the time of maximum concentration, variation in the exact time of sampling, and individual to individual variation in elimination rates. Lower Cmax/Cavg ratios mitigate each of these factors.

Larger window for titration or periodic evaluation sampling: With lower Cmax/Cavg ratios, the slower elimination curve makes possible the widening of the useable times for taking a sample, as the instantaneous T concentration is changing less rapidly, and titration can be based upon a sample taken over a range of times, e.g., 3 to 5 or 3 to 6 hours, demonstrated by the extension study.

More efficient conversion of prodrug dose to systemic T: A lower Cmax/Cavg ratio can be a characteristic of more efficient use of the taken dose. However, other factors also enter in, for instance a poorly absorbed dose would have minimal increase over the endogenous T level, and thus exhibit a low Cmax/Cavg ratio. But for a given absorbed (versus taken) dose, a lower Cmax ratio indicates a lengthening of effect and is desirable. A lower Cmax/Cavg ratio indicates that the fraction of time within the normal range of serum testosterone is higher.

### Advantages of Wider Dose Range

The development of a formulation of TU with a wide range of efficacious doses offers advantages, including:

A wider patient population can be treated: It is well-known that individuals absorb TU and convert TU to T to different degrees and that some individuals require a much lower dose than others, while others require a higher dose. For example, in the 2019EXT trial, out of 130 subjects at Day 90, 3 were at a dose level of 100 mg once daily (25% of the starting dose of 400 mg daily), and another 8 at 200 mg daily dose (as 100 mg BID with meals). Thus, doses which were 50% or less of the starting dose allowed treatment of 8+3=11; 100%*11/130=8.5% of the subjects to be titrated. Without the wide dose range, these subjects may have had T-levels too high for the SOV2012-F1 formulation. Additionally, 33 of 130 (25%) subjects at Day 90 had been up-titrated to 800 mg total daily dose, which is 2× the starting daily dose of 400 mg. Thus, the wide dose range, extending down to 25% of the starting dose and up to 200% of the starting dose enabled over 30% of subjects to receive the appropriate dose. Without these wide dose range, some subjects would be at risk of either higher than physiological levels of T Cavg, sub-normal T Cavg levels, or Cmax levels above the FDA-described parameters.

Allowance for extrinsic factors: As TU absorption can also be affected by extrinsic factors such as diet and compliance, a wide range of doses allows individuals who might consume a sub-optimal diet with respect to TU absorption may still be titrated into the eugonadal range by access to the wide range of doses.

### Dose-Normalized Cavg

The dose normalized dose may be calculated as the average dose divided by the mean T Cavg. This attribute attempts to characterize the efficiency of a formulation in delivering the active substance. However, the clinical trial design will also affect the outcome of dose-normalized dose, in that for a formulation with a wide dose range, the dose normalized dose will vary widely across doses. The mean dose-normalized Cavg for T plasma was 0.69, and for serum 0.79. The differences reflect the known effects for measurement of T in the presence of TU in samples (LaChance).

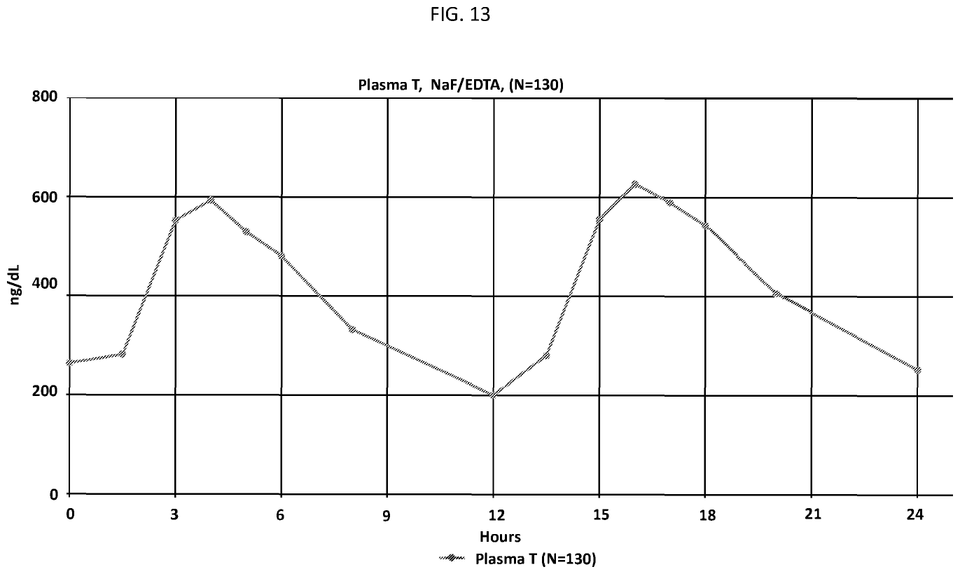

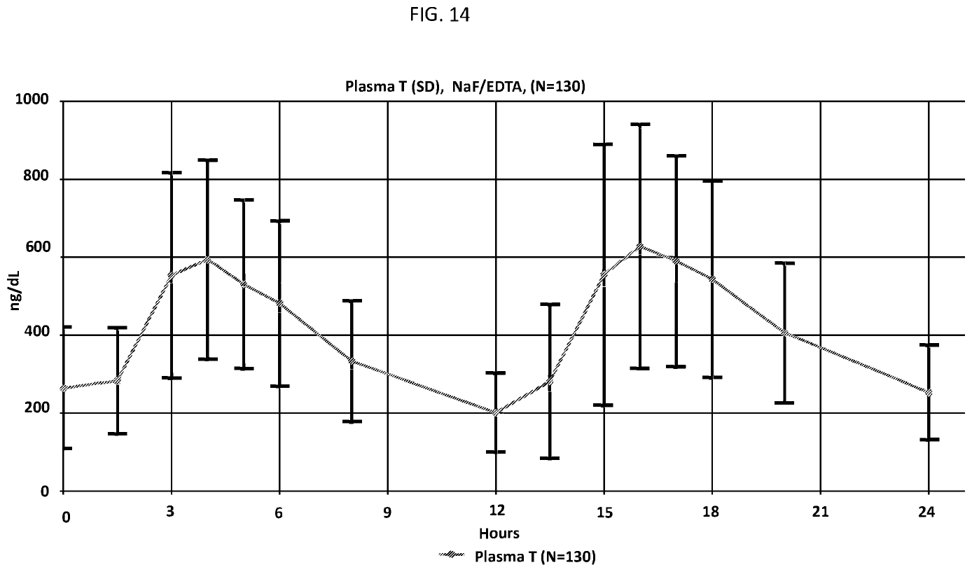

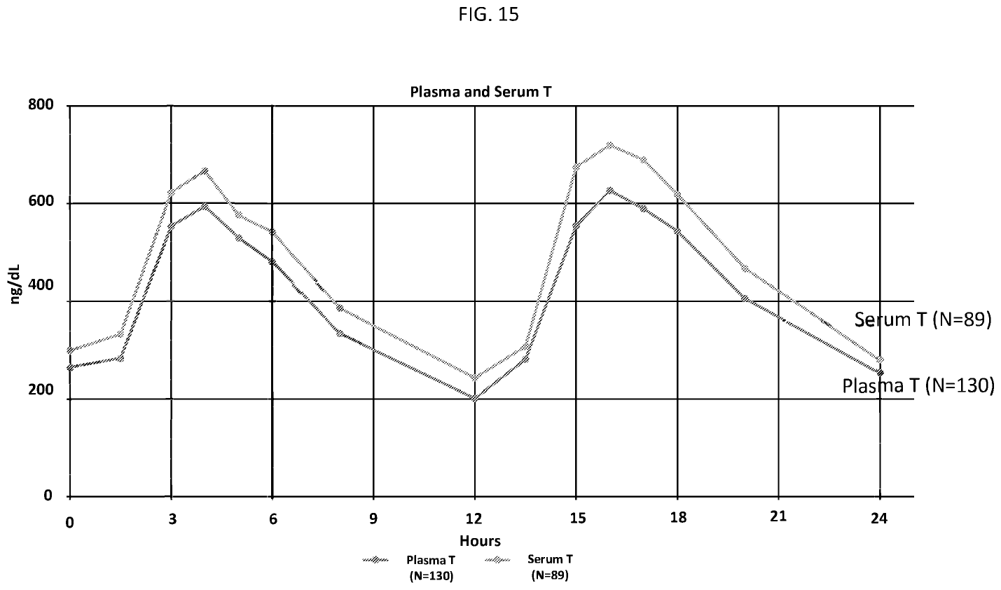

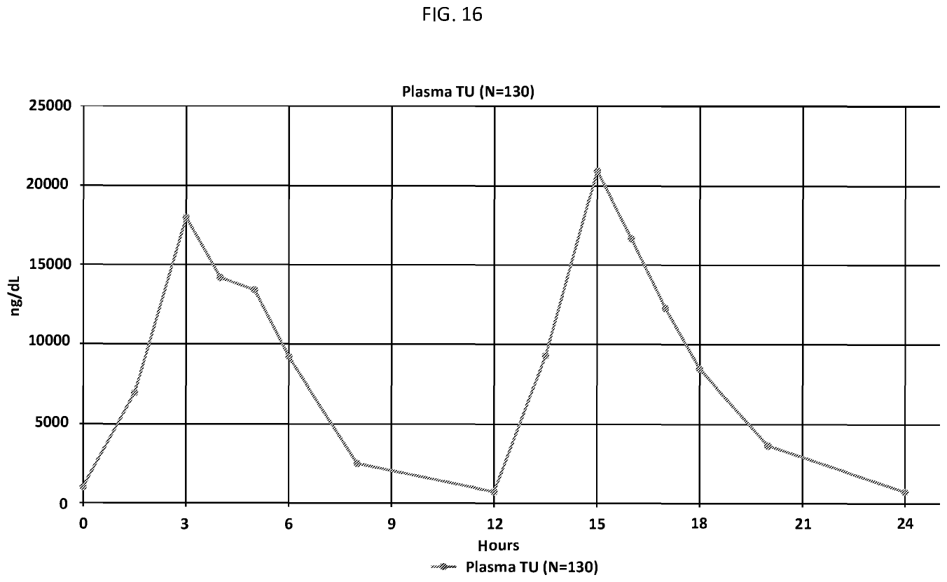

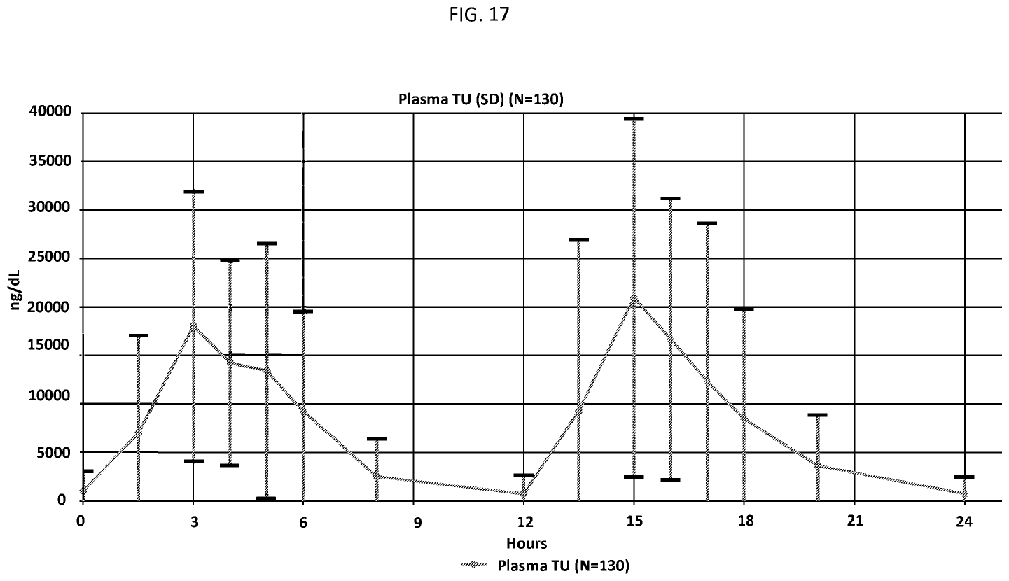

These data are summarized in Tables 12-14 and in FIGS. 13-17.

TABLE 12
Mean Pharmacokinetic Analyte Concentrations, Example 4

| | | | | | | | | Plasma | | | | | Plasma |
|---|---|---|---|---|---|---|---|---|---|---|---|---|---|
| | | | | | | | | DHT | | | | | DHTU |
| | | | | | | | | NaF/ | | | | | NaF/ |
| | | | | Plasma | Std | Serum | Std | EDT | Serum | Plasma | Plasma | | EDTA |
| | | | | T | Dev | T | Dev | A Tube | DHT | E2 | TU | Std Dev | Tube |
| Time- | Sta- | Visit | Time- | (N = | Plasma | (N = | Serum | (N = | (N = | (N = | (N = | Plasma | (N = |
| point | tistic | description | point | 130) | T | 89) | T | 130) | 130) | 130) | 130) | TU | 130) |
| Pre- | Mean | Visit 12E— | PRE | 264.13 | 156.241 | 298.36 | 178.211 | 66.79 | 68.52 | 3.20 | 1029.22 | 1993.401 | 1121.94 |
| dose | (SD) | Day 90E | DOSE | | | | | (49.760) | (45.892) | (2.141) | | | (1657.573) |
| 1.5 | Mean | Visit 12E— | 1.5 H | 282.15 | 136.084 | 331.49 | 149.726 | 56.94 | 59.14 | 2.77 | 6956.86 | 10005.583 | 1828.15 |
| Hours | (SD) | Day 90E | POST | | | | | (30.700) | (29.351) | (1.696) | | | (2236.755) |
| Post- | | | DOSE | | | | | | | | | | |
| dose | | | | | | | | | | | | | |
| 3 | Mean | Visit 12E— | 3 H | 552.81 | 263.913 | 621.77 | 267.831 | 81.23 | 84.09 | 3.03 | 17990.7 | 13932.535 | 7081.33 |
| Hours | (SD) | Day 90E | POST | | | | | (38.700) | (35.849) | (1.513) | | | (5633.424) |
| Post- | | | DOSE | | | | | | | | | | |
| dose | | | | | | | | | | | | | |
| 4 | Mean | Visit 12E— | 4 H | 593.31 | 255.922 | 666.26 | 284.343 | 96.80 | 101.96 | 3.38 | 14190.56 | 10539.604 | 7592.21 |
| Hours | (SD) | Day 90E | POST | | | | | (43.568) | (42.921) | (1.716) | | | (5776.677) |
| Post- | | | DOSE | | | | | | | | | | |
| dose | | | | | | | | | | | | | |
| 5 | Mean | Visit 12E— | 5 H | 529.5 | 217.39 | 575.7 | 234.233 | 104.08 | 104.24 | 3.39 | 13411.03 | 13105.362 | 7929.10 |
| Hours | (SD) | Day 90E | POST | | | | | (52.101) | (47.011) | (1.763) | | | (6245.538) |
| Post- | | | DOSE | | | | | | | | | | |
| dose | | | | | | | | | | | | | |
| 6 | Mean | Visit 12E— | 6 H | 480.78 | 212.076 | 541.71 | 241.769 | 104.40 | 103.43 | 3.43 | 9206.16 | 10247.981 | 7017.52 |
| Hours | (SD) | Day 90E | POST | | | | | (55.105) | (47.394) | (1.832) | | | (6857.650) |
| Post- | | | DOSE | | | | | | | | | | |
| dose | | | | | | | | | | | | | |
| 8 | Mean | Visit 12E— | 8 H | 332.46 | 155.014 | 384.73 | 218.566 | 91.76 | 91.31 | 3.46 | 2511.36 | 3900.573 | 3158.91 |
| Hours | (SD) | Day 90E | POST | | | | | (52.704) | (49.295) | (2.024) | | | (4111.955) |
| Post- | | | DOSE | | | | | | | | | | |
| dose | | | | | | | | | | | | | |
| 12 | Mean | Visit 12E— | 12 H | 200.76 | 101.178 | 242.54 | 160.875 | 55.03 | 54.65 | 3.20 | 734.6 | 1911.645 | 857.97 |
| Hours | (SD) | Day 90E | POST | | | | | (32.665) | (33.883) | (2.468) | | | (1918.190) |
| Post- | | | DOSE | | | | | | | | | | |
| dose | | | | | | | | | | | | | |
| 13.5 | Mean | Visit 12E— | 13.5 H | 280.54 | 196.682 | 307 | 183.894 | 54.64 | 53.05 | 3.01 | 9294.11 | 17595.989 | 2283.61 |
| Hours | (SD) | Day 90E | POST | | | | | (29.963) | (28.043) | (2.062) | | | (3578.808) |
| Post- | | | DOSE | | | | | | | | | | |
| dose | | | | | | | | | | | | | |
| 15 | Mean | Visit 12E— | 15 H | 554.67 | 334.694 | 675.02 | 353.465 | 79.43 | 83.79 | 3.22 | 20950.57 | 18437.455 | 8007.25 |
| Hours | (SD) | Day 90E | POST | | | | | (43.240) | (43.097) | (1.942) | | | (7407.737) |
| Post- | | | DOSE | | | | | | | | | | |
| dose | | | | | | | | | | | | | |
| 16 | Mean | Visit 12E— | 16 H | 626.74 | 313.118 | 719.49 | 331.007 | 99.01 | 102.34 | 3.73 | 16679.08 | 14483.901 | 8658.61 |
| Hours | (SD) | Day 90E | POST | | | | | (52.248) | (49.174) | (2.003) | | | (6810.861) |
| Post- | | | DOSE | | | | | | | | | | |
| dose | | | | | | | | | | | | | |
| 17 | Mean | Visit 12E— | 17 H | 589.25 | 271.13 | 689.4 | 307.183 | 109.10 | 112.37 | 4.10 | 12289.71 | 16342.213 | 7570.84 |
| Hours | (SD) | Day 90E | POST | | | | | (54.758) | (50.703) | (2.233) | | | (6767.135) |
| Post- | | | DOSE | | | | | | | | | | |
| dose | | | | | | | | | | | | | |
| 18 | Mean | Visit 12E— | 18 H | 543.2 | 253.06 | 618.43 | 289.303 | 109.65 | 113.57 | 4.35 | 8484.73 | 11275.579 | 5886.93 |
| Hours | (SD) | Day 90E | POST | | | | | (53.031) | (53.403) | (2.328) | | | (6088.431) |
| Post- | | | DOSE | | | | | | | | | | |
| dose | | | | | | | | | | | | | |
| 20 | Mean | Visit 12E— | 20 H | 405.34 | 179.436 | 467.11 | 217.401 | 97.64 | 100.61 | 4.48 | 3642.56 | 5195.894 | 3135.54 |
| Hours | (SD) | Day 90E | POST | | | | | (51.143) | (49.757) | (2.493) | | | (3330.261) |
| Post- | | | DOSE | | | | | | | | | | |
| dose | | | | | | | | | | | | | |
| 24 | Mean | Visit 12E— | 24 H | 252.36 | 120.574 | 278.93 | 120.332 | 66.80 | 67.04 | 3.95 | 741.27 | 1752.157 | 818.56 |
| Hours | (SD) | Day 90E | POST | | | | | (45.348) | (41.128) | (2.461) | | | (1486.923) |
| Post- | | | DOSE | | | | | | | | | | |
| dose | | | | | | | | | | | | | |

TABLE 13
Mean Cmax, Cavg, Cmax/C0, Cmax/C12
and Cmax/Cavg ratios, Example 4

| Means | |
|---|---|
| Mean Cmax 0-24 (n = 130) | 852.37 |
| Ratio Cmax to C12 | 4.25 |
| Ratio Cmax to C0 | 3.23 |
| Mean Cmax 0-12 | 695.85 |
| Ratio Cmax 0-12 to C12 | 3.47 |
| Ratio Cmax 0-12 to C0 | 2.63 |
| Cavg 0-12 | 374.84 |
| Ratio Cmax 0-12/Cavg 0- | 1.86 |
| 12 | |
| Mean Cmax 12-24 | 755.95 |
| Ratio Cmax 12-24 to C12 | 3.77 |
| Ratio Cmax 12-24 to C0 | 2.86 |
| Cavg 12-24 | 413.82 |
| Ratio Cmax 12-24/Cavg | 1.83 |
| 12-24 | |
| Mean Cavg 0-24 (n = 127) | 393.29 |
| Cmax 0-24/Cavg 0-24 | 2.17 |

TABLE 14
Dose-normalized Cavg for Example 4, all values:

| Dose/day | | Plasma Dose- | Serum T | Serum Dose- |
|---|---|---|---|---|
| TU mg at Day | Plasma T | normal Cavg | Cavg | normal Cavg |
| 90 | Cavg | *10⁻⁶/dL | (n = 88) | *10⁻⁶/dL |
| Average dose | 393.3 | 0.69 | 451.92 | 0.79 |
| 571 mg | | | | |
| (n = 127 plasma; | | | | |
| n = 88 serum) | | | | |
| 100 mg | 424 | 4.24 | 545.0 | 5.45 |
| 200 mg | 407.8 | 2.04 | 450.7 | 2.25 |
| 400 mg | 383.7 | 0.96 | 433.5 | 1.08 |
| 600 mg | 404.5 | 0.67 | 454.9 | 0.76 |
| 800 mg | 376.6 | 0.47 | 451.8 | 0.56 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A method of treating testosterone deficiency in a subject in need thereof, the method comprising:
a) performing a treatment regimen comprising orally administering to the subject 400 mg of testosterone undecanoate (TU) daily with a meal, wherein the TU is administered in a pharmaceutical composition comprising TU, a non-sterol solubilizing agent effective for solubilization of the TU, and a phytosterol or phytosterol ester;
b) establishing a first steady state serum concentration of testosterone;
c) following step (b), providing a first steady state Serum Value of testosterone in the subject that is measured from about 3 hours to about 6 hours after administration of the pharmaceutical composition; and
d) performing a first titration of the testosterone undecanoate, wherein:
i) if the first Serum Value of testosterone is less than about 400/F+b ng/dL, then orally administering to the subject about 600 mg TU daily to establish a second steady state Serum Value of testosterone that is higher than the first steady state Serum Value of testosterone;
ii) if the first Serum Value of testosterone is from about 400/F+b ng/dL to about 900/F+b ng/dL, then continuing to orally administer to the subject about 400 mg TU daily to maintain the first steady state Serum Value of testosterone; or
iii) if the first Serum Value of testosterone is greater than about 900/F+b ng/dL, then orally administering to the subject about 200 mg TU daily to establish a second steady state Serum Value of testosterone that is lower than the first steady state Serum Value of testosterone.

2. The method of claim 1, wherein step (a) comprises administering the pharmaceutical composition twice daily.

3. The method of claim 2, wherein a first dose is administered in the morning and a second dose is administered in the evening.

4. The method of claim 2 or 3, wherein the first dose comprises about 200 mg TU, and the second dose comprises about 200 mg TU.

5. The method of claim 4, wherein following the first titration,
i) about 600 mg TU is administered daily to the subject, and the first dose comprises about 300 mg TU, and the second dose comprises about 300 mg TU;
ii) about 400 mg TU is administered daily to the subject, and the first dose comprises about 200 mg TU, and the second dose comprises about 200 mg TU; or
iii) about 200 mg TU is administered daily to the subject, and the first dose comprises about 100 mg TU, and the second dose comprises about 100 mg TU.

6. The method of any one of claims 1-5, further comprising:
e) establishing a second steady state serum concentration of testosterone;
f) following step (e), providing a second steady state Serum Value of testosterone in the subject; and
g) performing a second titration of the TU.

7. The method of claim 6, wherein following the first titration, about 600 mg TU is administered daily to the subject, and
a) if the second Serum Value of testosterone is less than about 400/F+b ng/dL, then orally administering about 800 mg TU daily to the subject to establish a third steady state Serum Value of testosterone that is higher than the second steady state Serum Value of testosterone;
b) if the second Serum Value of testosterone is from about 400/F+b ng/dL to about 900/F+b ng/dL, then continuing to orally administer about 600 mg TU daily to the subject to maintain the second steady state Serum Value of testosterone; or
c) if the second Serum Value of testosterone is greater than about 900/F+b ng/dL, then orally administering about 400 mg TU daily to the subject to establish a third steady state Serum Value of testosterone that is lower than the second steady state Serum Value of testosterone.

8. The method of claim 6, wherein following the first titration, about 400 mg TU is administered daily to the subject, and
a) if the second Serum Value of testosterone is less than about 400/F+b ng/dL, then orally administering about 600 mg TU daily to the subject to establish a third steady state Serum Value of testosterone that is higher than the second steady state Serum Value of testosterone;
b) if the second Serum Value of testosterone is from about 400/F+b ng/dL to about 900/F+b ng/dL, then continuing to orally administer about 400 mg TU daily to the subject to maintain the second steady state Serum Value of testosterone; or
c) if the second Serum Value of testosterone is greater than about 900/F+b ng/dL, orally administering about 200 mg TU daily to the subject to establish a third steady state Serum Value of testosterone that is lower than the second steady state Serum Value of testosterone; or

9. The method of claim 6, wherein following the first titration, about 200 mg TU is administered daily to the subject, and
a) if the second Serum Value of testosterone is less than about 400/F+b ng/dL, orally administering about 400 mg TU daily to the subject to establish a third steady state Serum Value of testosterone that is higher than the second steady state Serum Value of testosterone;
b) if the second Serum Value of testosterone is from about 400/F+b ng/dL to about 900/F+b ng/dL, then continuing to orally administer about 200 mg TU daily to the subject to maintain the second steady state Serum Value of testosterone; or
c) if the second Serum Value of testosterone is greater than about 900/F+b ng/dL, orally administering about 100 mg TU daily to the subject to establish a third steady state Serum Value of testosterone that is lower than the second steady state Serum Value of testosterone.

10. The method of claim 7, wherein following the second titration, about 800 mg TU is administered daily to the subject, and the first dose comprises about 400 mg TU, and the second dose comprises about 400 mg TU.

11. The method of claim 9, wherein following the second titration, about 100 mg TU is administered daily to the subject, and the subject receives a single dose of about 100 mg TU.

12. The method of claim 11, wherein the single dose of about 100 mg TU is administered in the morning.

13. The method of any one of claims 1-12, wherein the first Serum Value of testosterone is measured prior to day 21 of the treatment regimen.

14. The method of claim 13, wherein the first Serum Value of testosterone is measured on about day 7 of the treatment regimen.

15. The method of any one of claims 1-14, wherein the first titration is performed on from about day 7 to about day 35 of the treatment regimen.

16. The method of claim 15, wherein the first titration is performed on about day 28 of the treatment regimen.

17. The method of any one of claims 1-16, wherein the pharmaceutical composition comprises from about 5% to about 40% by weight testosterone undecanoate.

18. The method of any one of claims 1-17, wherein the pharmaceutical composition comprises from about 2% to about 45% by weight of a phytosterol or phytosterol ester.

19. The method of any one of claims 1-18, wherein the pharmaceutical composition comprises from about 10% to about 90% by weight of a non-sterol solubilizing agent.

20. The method of any one of claims 1-19, wherein the non-sterol solubilizing agent is selected from lipids, surfactants, and mixtures thereof.

21. The method of any one of claims 1-19, wherein the non-sterol solubilizing agent comprises propylene glycol monolaurate.

22. The method of any one of claims 1-19, wherein the non-sterol solubilizing agent comprises polyoxyl 40 hydrogenated castor oil.

23. The method of any one of claims 1-22, wherein the pharmaceutical composition is self-emulsifying or self-microemulsifying.

24. The method of any one of claims 1-23, wherein the pharmaceutical composition comprises phytosterol esters.

25. The method of any one of claims 1-24, wherein the pharmaceutical composition comprises:
a) from about 10% to about 25% by weight of solubilized testosterone undecanoate;
b) from about 5% to about 40% by weight of a hydrophilic surfactant;
c) from about 15% to about 65% by weight of a hydrophobic surfactant;
d) from about 2% to about 45% by weight of phytosterol esters; and
e) from about 0 to about 15% by weight of a solubilizer.

26. The method of claim 25, wherein the pharmaceutical composition comprises from about 10% to about 40% by weight of one or more phytosterol esters.

27. The method of claim 26, wherein the pharmaceutical composition comprises from about 10% to about 30% by weight of one or more phytosterol esters.

28. The method of any one of claims 25-27, wherein the solubilizer comprises dl-alpha-tocopherol and/or an ester or acetate thereof.

29. The method of any one of claims 25-28, wherein the pharmaceutical composition comprises:
a) about 18.2% by weight of solubilized testosterone undecanoate;
b) about 15.0% by weight of polyoxyl 40 hydrogenated castor oil;
c) about 39.9% by weight of propylene glycol monolaurate;
d) about 25.0% by weight of one or more phytosterol esters; and
e) about 2.0% by weight of dl-alpha-tocopherol and/or an ester or acetate thereof.

30. The method of any one of claims 1-29, wherein the subject is a hypogonadal male.

31. The method of any one of claims 1-30, wherein the subject has not previously been administered TU or other testosterone replacement therapy for a period of at least seven days or a period of time sufficient to completely wash exogenous testosterone from the subject.

32. The method of any one of claims 1-31, wherein the method is performed on a population of human subjects.

33. The method of claim 32, wherein the population comprises at least 10 subjects, at least 50 subjects, at least 100 subjects, at least 200 subjects, at least 500 subjects, or more.

34. The method of claim 32 or 33, wherein the method:
a) achieves a Cavg in the serum normal range of about 300 ng/dL to about 1000 ng/dL in at least 75% of the population;
b) achieves a Cmax of less than about 1500 ng/dL in at least 85% of the population;
c) achieves a Cmax of from about 1800 ng/dL to about 2500 ng/dL in no more than 5% of the population; and/or
d) achieves a Cmax of greater than about 2500 ng/dL in no more than 0% of the population.

35. The method of any one of claims 32-34, wherein the method reduces an average number of incorrect titrations or the risk of incorrect titrations per subject in the population in order to achieve a steady state testosterone Serum Value of from about 300 ng/dL to about 1000 ng/dL relative to a population receiving a treatment regimen in which an initial dosage is not about 400 mg TU and/or the Serum Value is not measured from about 3 hours to about 6 hours after administration.

36. The method of any one of claims 32-35, wherein the method:
a) achieves a Cavg in the serum normal range of about 300 ng/dL to about 1000 ng/dL in a greater number of subjects in the population as compared to a treatment regimen in which an initial dosage is not about 400 mg TU and/or the Serum Value is not measured from about 3 hours to about 6 hours after administration;
b) achieves a Cmax of less than about 1500 ng/dL in a greater number of subjects in the population as compared to the treatment regimen in which the initial dosage is not about 400 mg TU and/or the Serum Value is not measured from about 3 hours to about 6 hours after administration;
c) achieves a Cmax of from about 1800 ng/dL to about 2500 ng/dL in a fewer number of subjects in the population as compared to the treatment regimen in which the initial dosage is not about 400 mg TU and/or the Serum Value is not measured from about 3 hours to about 6 hours after administration; and/or
d) achieves a Cmax of greater than about 2500 ng/dL in a fewer number of subjects in the population as compared to the treatment regimen in which the initial dosage is not about 400 mg TU and/or the Serum Value is not measured from about 3 hours to about 6 hours after administration.

37. The method of any one of claims 32-36, wherein the method decreases the risk of elevated blood pressure of the population of human subjects.

38. The method of claim 37, wherein the daytime systolic blood pressure, night time systolic blood pressure, and/or 24-hour average systolic blood pressure does not increase by more than 3 mmHg as compared to the blood pressure before onset of treatment in the population of human subjects.

39. The method of claim 37, wherein the subjects are diabetic or hypertensive and the daytime systolic blood pressure, night time systolic blood pressure, and/or 24-hour average systolic blood pressure does not increase by more than 4 mmHg as compared to the blood pressure before onset of treatment in the population of human subjects.

40. The method of any one of claims 32-39, wherein the population averages has:
a) a Cmax/Cavg ratio for 0-24 hours of less than 2.5;
b) a Cmax/Cavg ratio for 0-12 hours of less than 2.2; and/or
c) a Cmax/Cavg ratio for 12-24 hours of less than 2.2.

41. The method of any one of claims 1-40, wherein the first Serum Value is measured by:
(a) measuring testosterone concentration of serum clotted at room temperature for about 30 minutes prior to centrifugation in a tube;
(b) measuring testosterone concentration of plasma in a tube supplemented with EDTA and NaF and multiplying the testosterone concertation by the inverse of a predetermined factor F (1/F); or
(c) a comparable method thereof.

42. The method of any one of claims 6-41, wherein the second Serum Value is measured by:
(a) measuring testosterone concentration of serum clotted at room temperature for about 30 minutes prior to centrifugation in a tube;
(b) measuring testosterone concentration of plasma in a tube supplemented with EDTA and NaF and multiplying the testosterone concentration by the inverse of a predetermined factor F (1/F); or
(c) a comparable method thereof.

43. The method of claim 41 or 42, wherein the predetermined factor F is from about 0.70 to about 1.10.

44. The method of claim 43, wherein the predetermined factor F is from about 0.81 to about 0.94.

45. The method of claim 44, wherein the predetermined factor F is about 0.89.

46. The method of any one of claims 1-45, wherein the subject is at risk of high blood pressure, heart attack, or stroke.

47. The method of any one of claims 1-46, wherein the subject is suffering from low testosterone levels due to aging.

48. The method of any one of claims 1-47, wherein the subject is suffering from low testosterone levels due to a disease which decreases testosterone production.

49. The method of any one of claims 1-48, wherein the subject has diabetes, hypertension, a metabolic disorder, or is obese.

50. The method of claim 49, wherein the diabetes is diabetes mellitus.

51. The method of any one of claims 1-50, wherein the subject has previously been treated with an anti-hypertensive medication.

52. The method of any one of claims 1-51, wherein the subject has osteoporosis, reduced sexual function or libido, muscle strength or muscle stamina, aplastic anemia, AIDS wasting syndrome, obstructive sleep apnea, metabolic disorders, non-alcoholic fatty liver disease (NAFLD), or non-alcoholic steatohepatitis (NASH).

53. The method of any one of claims 1-52, wherein the subject is at risk of a testosterone related adverse event.

54. A method of treating testosterone deficiency in a subject in need thereof, the method comprising:
a) performing a treatment regimen comprising orally administering to the subject 400 mg of testosterone undecanoate (TU) daily with a meal, wherein the TU is administered in a pharmaceutical composition comprising TU, a non-sterol solubilizing agent effective for solubilization of the TU, and a phytosterol or phytosterol ester;
b) establishing a first steady state serum concentration of testosterone;
c) following step (b), providing a first steady state Serum Value of testosterone in the subject that is measured from about 3 hours to about 6 hours after administration of the pharmaceutical composition; and
d) performing a first titration of the testosterone undecanoate, wherein:
i) if the first Serum Value of testosterone is less than about 460 ng/dL, then orally administering to the subject about 600 mg TU daily to establish a second steady state Serum Value of testosterone that is higher than the first steady state Serum Value of testosterone;
ii) if the first Serum Value of testosterone is from about 460 ng/dL to about 971 ng/dL, then continuing to orally administer to the subject about 400 mg TU daily to maintain the first steady state Serum Value of testosterone; or
iii) if the first Serum Value of testosterone is greater than about 971 ng/dL, then orally administering to the subject about 200 mg TU daily to establish a second steady state Serum
Value of testosterone that is lower than the first steady state Serum Value of testosterone; wherein the subject:
is on anti-hypertensive therapy and exhibits an average change in systolic blood pressure of no more than 3.4 mmHg, an average change in diastolic blood pressure of no more than 1.8 mmHg, and/or an average change in heart rate of no more than 1.3 beats per minute; and/or
has diabetes mellitus and exhibits an average change in systolic blood pressure of no more than 3.0 mmHg, an average change in diastolic blood pressure of no more than 1.7 mmHg, and/or an average change in heart rate of no more than 1.9 beats per minute.
